SEQUENCE LISTING (1 sequence)

SEQ ID NO: 1 - protein, 19 aa, Homo Sapiens
DLDLEMLAPYIPMDDDFQL

(19) United States
(12) Patent Application Publication
(10) Pub. No.: US 2017/0240513 A1
(43) Pub. Date: Aug. 24, 2017

(54) SUBSTITUTED PYRIMIDINES AS INHIBITORS OF HIF PROLYL HYDROXYLASE

(71) Applicants: FEZ UJJAINWALLA, KENILWORTH, NJ (US); JOHN QIANG TAN, PRINCETON, NJ (US); QUN DANG, KENILWORTH, NJ (US); CHRISTOPHER J. SINZ, RAHWAY, NJ (US); MING WANG, RAHWAY, NJ (US); YILI CHEN, RAHWAY, NJ (US); JIAQIANG CAI, SHANGHAI (CN); MERCK SHARP & DOHME CORP., RAHWAY, NJ (US)

(72) Inventors: FEZ UJJAINWALLA, HOBOKEN, NJ (US); JOHN QIANG TAN, PRINCETON, NJ (US); QUN DANG, WESTFIELD, NJ (US); CHRISTOPHER J. SINZ, MIDDLETOWN, NJ (US); MING WANG, BELLE MEAD, NJ (US); YILI CHEN, HILLSBOROUGH, NJ (US); JIAQIANG CAI, SHANGHAI (CN)

(73) Assignee: MERCK SHARP & DOHME CORP., RAHWAY, NJ (US)

(21) Appl. No.: 15/517,550

(22) PCT Filed: Oct. 8, 2015
(86) PCT No.: PCT/US15/54626

§ 371 Date: Apr. 7, 2017

(30) Foreign Application Priority Data
Oct. 10, 2014 (CN) PCT/CN2014/088319

(51) Int. Cl. C07D 239/36; C07D 413/06

(52) U.S. Cl. CPC C07D 239/36; C07D 413/06

(57) ABSTRACT

The present invention concerns compounds of Formula I that inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### BACKGROUND OF THE INVENTION

The insufficient delivery of oxygen to cells and tissues is associated with anemia, which is defined as a deficiency in the blood's oxygen-carrying capacity, and ischemia, in which restrictions in blood supply are caused by a constriction or blockage of blood vessels. Anemia can be caused by the loss of red blood cells (hemorrhage), excessive red blood cell destruction (hemolysis) or deficiencies in erythropoiesis (production of red blood cells from precursors found in the bone marrow). The symptoms of anemia can include weakness, dizziness, fatigue, pallor, impairment of cognitive function and a general reduction in quality of life. Chronic and/or severe anemia can lead to the exacerbation of myocardial, cerebral or peripheral ischemia and to heart failure. Ischemia is defined as an absolute or relative shortage of oxygen to a tissue or organ and can result from disorders such as atherosclerosis, diabetes, thromboembolisms, hypotension, etc. The heart, brain and kidney are especially sensitive to ischemic stress caused by low blood supply.

The primary pharmacological treatment for anemia is administration of some variant of recombinant human erythropoietin (EPO). For anemias associated with kidney disease, chemotherapy-induced anemia, anemia from HIV-therapy or anemia due to blood loss, recombinant EPO is administered to enhance the supply of the hormone, correct the shortage of red blood cells and increase the blood's oxygen-carrying capacity. EPO replacement is not always sufficient to stimulate optimal erythropoiesis (e.g., in patients with iron processing deficiencies) and has associated risks.

Hypoxia-inducible factor (HIF) has been identified as a primary regulator of the cellular response to low oxygen. HIF is a heterodimeric gene transcription factor consisting of a highly regulated α-subunit (HIF-α) and a constitutively expressed β-subunit (HIF-β, also known as ARNT, or aryl hydrocarbon receptor nuclear transporter). HIF target genes are reported to be associated with various aspects of erythropoiesis (e.g., erythropoietin (EPO) and EPO receptor), glycolysis and angiogenesis (e.g., vascular endothelial growth factor (VEGF)). Genes for proteins involved in iron absorption, transport and utilization as well as heme synthesis are also targets of HIF.

Under normal oxygenation, HIF-α is a substrate in a reaction with molecular oxygen, which is catalyzed by a family of iron(II)-, 2-ketoglutarate- and ascorbate-dependent dioxygenase enzymes called PHD-1 (EGLN2, or egg laying abnormal 9 homolog 2, PHD2 (EGLN1), and PHD3 (EGLN3). Proline residues of HIF-α are hydroxylated (e.g., Pro-402 and Pro-564 of HIF-la) and the resulting product is a target of the tumor suppressor protein von-Hippel Lindau, a component of an E3 ubiquitin ligase multiprotein complex involved in protein ubiquitination. Under low oxygenation, the HIF-α hydroxylation reaction is less efficient and HIF-α is available to dimerize with HIF-β. HIF dimers are translocated to the cell nucleus where they bind to a hypoxia-responsive enhancer element of HIF target genes.

Cellular levels of HIF are known to increase under conditions of hypoxia and after exposure to hypoxia mimetic agents. The latter includes, but is not limited to, specific metal ions (e.g., cobalt, nickel, manganese), iron chelators (e.g., desferrioxamine) and analogs of 2-ketoglurate (e.g., N-oxalyl glycine). The compounds of the present invention inhibit the HIF prolyl hydroxylases (PHD-1, PHD-2, PHD-3) and can also serve to modulate HIF levels. These compounds therefore have utility for the treatment and/or prevention of disorders or conditions where HIF modulation is desirable, such as anemia and ischemia. As an alternative to recombinant erythropoietin therapy, the compounds of the present invention provide a simpler and broader method for the management of anemia.

### SUMMARY OF THE INVENTION

The present invention concerns compounds of formula I

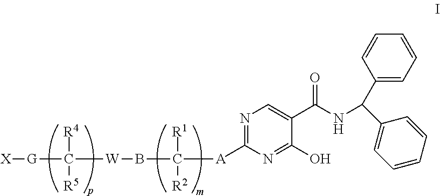

which inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### DETAILED DESCRIPTION OF THE INVENTION

The present invention provides compounds of formula I or stereoisomers or pharmaceutically acceptable salts thereof:

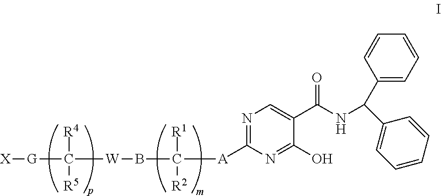

A is —C(O)NR⁹—, or —C(O)—;
B is selected from a bond, C₃₋₁₂cycloalkyldiyl and C₃₋₁₂cycloheteroalkyldiyl, wherein B is optionally substituted by 0 or 1 oxo, C₁₋₆ alkyl, or halogen;
W is a bond or —C(O)O—;
G is selected from a bond, —OC(O)—, —OC(O)O—, —C(O)—, and —C(O)O—;
X is selected from hydrogen, C₁₋₁₀ alkyl, arylC₀₋₅ alkyl, C₃₋₁₂cycloalkylC₀₋₅ alkyl, C₃₋₁₂ heterocycloalkylC₀₋₅ alkyl, and —NR⁶R⁷, wherein X is substituted with 0, 1, or 2 halogen, C₁₋₃alkyl, hydroxyC₁₋₃alkyl, hydroxy, oxo, C₁₋₆haloalkyl, or C₁₋₃alkoxy;
R¹ and R² are each independently selected from hydrogen, C₁₋₃alkyl, hydroxyC₁₋₃alkyl, and hydroxy, wherein R¹ and R² may optionally join together with the carbon to which they are attached to form a 3 to 8 membered saturated ring;
R⁹ is selected from hydrogen, C₁₋₃alkyl, hydroxyC₁₋₃alkyl, hydroxy, or C₁₋₃alkoxy;
p is 0, 1, or 2;
m is 0, 1, or 2;
R⁶ and R⁷ are each independently selected from hydrogen, C₁₋₆ alkyl, aryl, and C₁₋₆ haloalkyl;
R⁴ and R⁵ are each independently selected from

hydrogen,

halogen,

C₁₋₁₀ alkyl,

C₂₋₁₀ alkenyl,

C₂₋₁₀ alkynyl,

C₁₋₁₀ alkylamino,

arylC₀₋₁₀ alkyl,

C₃₋₈ cycloalkyl C₀₋₁₀ alkyl,

C₃₋₈ heteroaryl C₀₋₁₀ alkyl,

C₃₋₈ heterocycloalkyl C₀₋₁₀ alkyl,

C₁₋₁₀ alkoxy, and

hydroxyC₀₋₁₀alkyl, wherein R⁴ and R⁵ may optionally join together with the carbon to which they are attached to form a 3 to 8 membered ring; and

wherein R⁴ and R⁵ are each optionally substituted with 0, 1, or 2 R⁸ substituents selected from:

hydrogen,

halogen,

hydroxy,

(carbonyl)₀₋₁C₁₋₁₀ alkyl,

amino C₀₋₁₀ alkyl,

C₁₋₁₀ alkylamino C₀₋₁₀ alkyl,

cyano,

nitro,

C₁₋₆haloalkyl,

perfluoroC₁₋₆alkyl, and

perfluoroC₁₋₆alkoxy; and

provided that when B and W are bonds, p is 0, and G is —C(O)O—, then X is other than hydrogen; and provided that when B is a bond, W is —C(O)O—, p is 0, and G is a bond, than X is other than hydrogen.

Another embodiment of the invention provides compounds of Formula II or stereoisomers thereof, or pharmaceutically acceptable salts thereof:

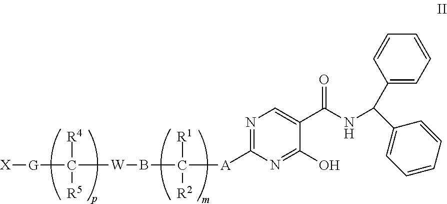

A is —C(O)NH—, or —C(O)—;
B is selected from a bond, C₃₋₁₂cycloalkyldiyl and C₃₋₁₂cycloheteroalkyldiyl, wherein B is optionally substituted by 0 or 1 oxo, C₁₋₆ alkyl, or halogen;
W is a bond or —C(O)O—;
G is selected from a bond, —OC(O)—, —OC(O)O—, —C(O)—, and —C(O)O—;
X is selected from hydrogen, C₁₋₁₀ alkyl, arylC₀₋₅ alkyl, C₃₋₁₂cycloalkylC₀₋₅ alkyl, C₃₋₁₂ heterocycloalkylC₀₋₅ alkyl, and —NR⁶R⁷, wherein X is substituted with 0, 1, or 2 halogen, C₁₋₃alkyl, hydroxyC₁₋₃alkyl, hydroxy, oxo, C₁₋₆haloalkyl, or C₁₋₃alkoxy;
R¹ and R² are each independently selected from hydrogen, C₁₋₃alkyl, hydroxyC₁₋₃alkyl, and hydroxy, wherein R¹ and R² may optionally join together with the carbon to which they are attached to form a 3 to 8 membered saturated ring;
R⁹ is selected from hydrogen, C₁₋₃alkyl, hydroxyC₁₋₃alkyl, hydroxy, or C₁₋₃alkoxy;
p is 0, 1, or 2;
m is 0, 1, or 2;
R⁶ and R⁷ are each independently selected from hydrogen, C₁₋₆ alkyl, aryl, and C₁₋₆ haloalkyl;
R⁴ and R⁵ are each independently selected from

hydrogen,

halogen,

C₁₋₁₀ alkyl,

C₂₋₁₀ alkenyl,

C₂₋₁₀ alkynyl,

C₁₋₁₀ alkylamino,

arylC₀₋₁₀ alkyl,

C₃₋₈ cycloalkyl C₀₋₁₀ alkyl,

C₃₋₈ heteroaryl C₀₋₁₀ alkyl,

C₃₋₈ heterocycloalkyl C₀₋₁₀ alkyl,

C₁₋₁₀ alkoxy, and

hydroxyC₀₋₁₀alkyl, wherein R⁴ and R⁵ may optionally join together with the carbon to which they are attached to form a 3 to 8 membered ring; and

wherein R⁴ and R⁵ are each optionally substituted with 0, 1, or 2 R⁸ substituents selected from:

hydrogen,

halogen,

hydroxy,

(carbonyl)₀₋₁C₁₋₁₀ alkyl,

amino C₀₋₁₀ alkyl,

C₁₋₁₀ alkylamino C₀₋₁₀ alkyl,

cyano,

nitro,

C₁₋₆haloalkyl,

perfluoroC₁₋₆alkyl, and

perfluoroC₁₋₆alkoxy; and

and

provided that when B and W are bonds, p is 0, and G is —C(O)O—, then X is other than hydrogen; or when B is a bond, W is —C(O)O—, p is 0, and G is a bond, than X is other than hydrogen.

In one embodiment of the invention of the compounds of Formulas I and II, B is selected from a bond, cyclobutyldiyl, cyclopentyldiyl, cyclopropyldiyl, bicyclo[1.1.1]pentyldiyl, oxazinandiyl, and oxazolidindiyl, wherein B is optionally substituted by 0 or 1 oxo, C₁₋₆ alkyl, or halogen. In a variant of this embodiment, B is selected from a bond, cyclobutyldiyl, bicyclo[1.1.1]pentyldiyl, oxazinandiyl, and oxazolidindiyl, wherein B is optionally substituted by 0 or 1 oxo.

In yet another embodiment of the invention, B is a bond or cyclobutyldiyl. In a variant of this embodiment, B is a bond. In another variant of the embodiment, B is cyclobutyldiyl.

For clarification, when B and W are both bonds then —CR¹R²— is attached directly to —CR¹R²— In the instance when B and W are both bonds and p is 0, then —CR¹R²— is attached directly to G. Additionally, in the instance when B and W are both bonds, p is 0, and G is a bond, then —CR¹R²— is attached directly to X.

In one embodiment, in the compounds of Formulas I and II, R¹ and R² are each independently selected from hydrogen, C₁₋₃alkyl, and hydroxy, wherein R¹ and R² may optionally join together with the carbon to which they are both attached to form a 3 to 8 membered saturated ring.

In one embodiment of the invention, R⁴ and R⁵ are each independently selected from hydrogen, aryl, and C₁₋₁₀ alkyl.

In another embodiment of the compounds of Formula I and II, R⁴ and R⁵ are each independently selected from hydrogen, phenyl, methyl, ethyl, or propyl. In yet another embodiment of the invention, R⁴ and R⁵ are each independently selected from hydrogen, phenyl, and methyl.

In one embodiment of the compounds of Formulas I and II, W is a bond. In another embodiment, W is —C(O)O—.

In one embodiment of the invention of the compounds of Formulas I and II, G is a bond.

In another embodiment of the invention G is selected from —OC(O)—, —OC(O)O—, —C(O)—, and —C(O)O—.

In one embodiment of the invention of the compounds of Formulas I and II, X is selected from hydrogen, C₁₋₁₀ alkyl, arylC₀₋₅ alkyl, C₃₋₁₂cycloalkylC₀₋₅ alkyl, C₃₋₁₂ heterocycloalkylC₀₋₅ alkyl, and —NR⁶R⁷, wherein X is substituted with 0, 1, or 2 halogen, C₁₋₃alkyl, hydroxyC₁₋₃alkyl, hydroxy, oxo, C₁₋₆haloalkyl, or C₁₋₃alkoxy.

In another embodiment of the invention, X is selected from hydrogen, methyl, ethyl, propyl, isopropyl, phenyl, benzyl, cyclohexyl, 2,3-dihydro-1H-indenyl, dimethylamino, oxazinanyl, 1,3oxazinanyl, oxazolidinyl, and morpholinyl, wherein X is substituted with 0, 1, or 2 halogen, C₁₋₃alkyl, hydroxyC₁₋₃alkyl, hydroxy, oxo, C₁₋₆haloalkyl, or C₁₋₃alkoxy.

In yet another variant, X is selected from hydrogen, methyl, ethyl, propyl, isopropyl, phenyl, benzyl, cyclohexyl, 2,3-dihydro-1H-indenyl, dimethylamino, 1,3oxazinanyl, oxazolidinyl, and morpholinyl, wherein X is substituted with 0 or 1 hydroxy or oxo. In a variant of this embodiment, X is selected from hydrogen, methyl, ethyl, propyl, isopropyl, phenyl, benzyl, hydroxycyclohexyl, 2,3-dihydro-1H-indenyl, dimethylamino, 1,3oxazinanyl, oxazolidinyl, and morpholinyl, wherein X is substituted with 0 or 1 hydroxy or oxo.

In one embodiment of the compounds of Formulas I and II, R⁶ and R⁷ are each independently selected from hydrogen and C₁₋₆ alkyl.

In one embodiment of the compounds of Formula I and II, R⁸ substituents selected from: hydrogen, halogen, and hydroxy. In another embodiment R⁸ is hydrogen.

In one embodiment of the compounds of Formula I, R⁹ is selected from hydrogen or C₁₋₃alkyl. In another embodiment, R⁹ is selected from hydrogen.

In yet another embodiment of the invention provides compounds of Formula I or stereoisomers thereof, or pharmaceutically acceptable salts thereof:

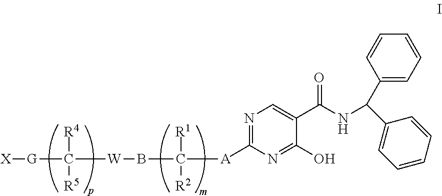

wherein
A is —C(O)NH—, or —C(O)—;
B is a bond or cyclobutyldiyl;
W is a bond or —C(O)O—;
G is selected from a bond, —OC(O)—, —OC(O)O—, —C(O)—, and —C(O)O—;
X is selected from methyl, ethyl, propyl, isopropyl, phenyl, benzyl, cyclohexyl, 2,3-dihydro-1H-indenyl, dimethylamino, oxazinanyl, oxazolidinyl, and morpholinyl, wherein X is substituted with 0 or 1 hydroxy or oxo;
   R¹ and R² are each independently selected from hydrogen, C₁₋₃alkyl, and hydroxy, wherein R¹ and R² may optionally join together with the carbon to which they are both attached to form a 3 to 8 membered saturated ring;
R⁹ is selected from hydrogen;
p is 0, 1, or 2;
m is 0, 1, or 2;
R⁶ and R⁷ are each independently selected from hydrogen and C₁₋₆ alkyl; and
R⁴ and R⁵ are each independently selected from hydrogen, aryl, and C₁₋₁₀ alkyl.

Representative compounds of the instant invention include, but are not limited to, the following compounds and their pharmaceutically acceptable salts and their stereoisomers thereof:
Methyl 3-(5-(benzhydrylcarbamoyl)-4-hydroxypyrimidine-2-carboxamido)propanoate;
2-morpholinoethyl 3-(5-(benzhydrylcarbamoyl)-4-hydroxypyrimidine-2-carboxamido)propanoate;
ethyl 1-((5-(benzhydrylcarbamoyl)-4-hydroxypyrimidine-2-carboxamido)methyl)cyclopropanecarboxylate;
ethyl 3-(5-(benzhydrylcarbamoyl)-4-hydroxypyrimidine-2-carboxamido)-2-hydroxy-2-methylpropanoate;
(R)-ethyl 3-(5-(benzhydrylcarbamoyl)-4-hydroxypyrimidine-2-carboxamido)-2-hydroxypropanoate;
Isopropyl 3-(5-(benzhydrylcarbamoyl)-4-hydroxypyrimidine-2-carboxamido)propanoate;
Acetoxymethyl 3-(5-(benzhydrylcarbamoyl)-4-hydroxypyrimidine-2-carboxamido)propanoate; ((isopropoxycarbonyl)oxy)methyl 3-(5-(benzhydrylcarbamoyl)-4-hydroxypyrimidine-2-carboxamido)propanoate;
1-((ethoxycarbonyl)oxy) ethyl 3-(5-(benzhydrylcarbamoyl)-4-hydroxypyrimidine-2-carboxamido)propanoate;
1-((isopropoxycarbonyl) oxy)ethyl 3-(5-(benzhydrylcarbamoyl)-4-hydroxypyrimidine-2-carboxamido)propanoate;
1-(((cyclohexyloxy) carbonyl)oxy)ethyl 3-(5-(benzhydrylcarbamoyl)-4-hydroxypyrimidine-2-carboxamido)propanoate;
((ethoxycarbonyl)oxy)methyl 3-(5-(benzhydrylcarbamoyl)-4-hydroxypyrimidine-2-carboxamido)propanoate;
2-(Dimethylamino)-2-oxoethyl 3-(5-(benzhydrylcarbamoyl)-4-hydroxypyrimidine-2-carboxamido)propanoate;
N-Benzhydryl-4-hydroxy-2-(6-oxo-1,3-oxazinane-3-carbonyl)pyrimidine-5-carboxamide;
2,3-Dihydro-1H-inden-5-yl 2-(5-(benzhydrylcarbamoyl)-4-hydroxypyrimidine-2-carboxamido)acetate;
2-(dimethylamino)-2-oxoethyl 2-(5-(benzhydrylcarbamoyl)-4-hydroxypyrimidine-2-carboxamido)acetate;
N-Benzhydryl-4-hydroxy-2-(5-oxooxazolidine-3-carbonyl)pyrimidine-5-carboxamide;
1-((Isopropoxycarbonyl)oxy)ethyl 3-(5-(benzhydrylcarbamoyl)-4-hydroxypyrimidin-2-yl)-3-oxopropanoate;
1-((ethoxycarbonyl)oxy)ethyl 2-(5-(benzhydrylcarbamoyl)-4-hydroxypyrimidine-2-carboxamido)acetate;
((Isopropoxycarbonyl)oxy)methyl 2-(5-(benzhydrylcarbamoyl)-4-hydroxypyrimidine-2-carboxamido)acetate;
(2-(5-(benzhydrylcarbamoyl)-4-hydroxypyrimidine-2-carboxamido)acetoxy)methyl isobutyrate;
(2-(5-(benzhydrylcarbamoyl)-4-hydroxypyrimidine-2-carboxamido)acetoxy)methyl propionate;
(2R)-1-(Benzyloxy)-1-oxopropan-2-yl 3-(5-(benzhydrylcarbamoyl)-4-hydroxypyrimidine-2-carboxamido)-2-hydroxypropanoate;
(2R)-2-ethoxy-2-oxo-1-phenylethyl 3-(5-(benzhydrylcarbamoyl)-4-hydroxypyrimidine-2-carboxamido)-2-hydroxypropanoate;
(2R)-2-Hydroxycyclohexyl 3-(5-(benzhydrylcarbamoyl)-4-hydroxypyrimidine-2-carboxamido)-2-hydroxypropanoate;
Ethyl 3-(5-(benzhydrylcarbamoyl)-4-hydroxypyrimidine-2-carboxamido)-2-hydroxy-2-methylpropanoate;
(Benzyl 3-(5-(benzhydrylcarbamoyl)-4-hydroxypyrimidine-2-carboxamido)propanoate;
(1R,3R)-2-isopropoxy-2-oxoethyl 3-(5-(benzhydrylcarbamoyl)-4-hydroxypyrimidine-2-carboxamido)cyclobutanecarboxylate; and
2-Isopropoxy-2-oxoethyl 3-(5-(benzhydrylcarbamoyl)-4-hydroxypyrimidine-2-carboxamido)-2-methylpropanoate.

As used herein except where noted, “alkyl” is intended to include both branched- and straight-chain saturated aliphatic hydrocarbon groups, including all isomers, having the specified number of carbon atoms. Commonly used abbreviations for alkyl groups are used throughout the specification, e.g. methyl may be represented by “Me” or CH₃, ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₆ alkyl” (or “C₁-C₆ alkyl”) for example, means linear or branched chain alkyl groups, including all isomers, having the specified number of carbon atoms. C₁₋₆ alkyl includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl.

The term “halogen” (or “halo”) refers to fluorine, chlorine, bromine and iodine (alternatively referred to as fluoro (F), chloro (Cl), bromo (Br), and iodo (I)).

The term “aryl” refers to aromatic mono- and poly-carbocyclic ring systems, wherein the individual carbocyclic rings in the polyring systems are fused or attached to each other via a single bond. Suitable aryl groups include phenyl, naphthyl, and biphenylenyl.

The term “carbocycle” (and variations thereof such as “carbocyclic” or “carbocyclyl”) as used herein, unless otherwise indicated, refers to (i) a C₃ to C₈ monocyclic, saturated or unsaturated ring or (ii) a C₇ to C₁₂ bicyclic saturated or unsaturated ring system. Each ring in (ii) is either independent of, or fused to, the other ring, and each ring is saturated or unsaturated. The carbocycle may be attached to the rest of the molecule at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A fused bicyclic carbocycle in which one ring is saturated and the other is saturated is a saturated bicyclic ring system. A fused bicyclic carbocycle in which one ring is benzene and the other is saturated is an unsaturated bicyclic ring system. A fused bicyclic carbocycle in which one ring is benzene and the other is unsaturated is an unsaturated ring system. Saturated carbocyclic rings are also referred to as cycloalkyl rings, e.g., cyclopropyl, cyclobutyl, etc. Unless otherwise noted, carbocycle is unsubstituted or substituted with C₁₋₆ alkyl, C₁₋₆ alkenyl, C₁₋₆ alkynyl, aryl, halogen, NH₂ or OH. A subset of the fused bicyclic unsaturated carbocycles are those bicyclic carbocycles in which one ring is a benzene ring and the other ring is saturated or unsaturated, with attachment via any carbon atom that results in a stable compound. Representative examples of this subset include the following:

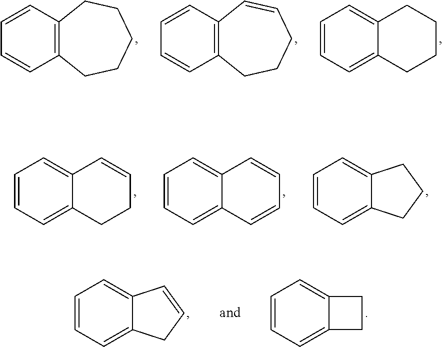

The term “heterocycle” (and variations thereof such as “heterocyclic” or “heterocyclyl”) broadly refers to (i) a stable 4- to 8-membered, saturated or unsaturated monocyclic ring, or (ii) a stable 7- to 12-membered bicyclic ring system, wherein each ring in (ii) is independent of, or fused to, the other ring or rings and each ring is saturated or unsaturated, and the monocyclic ring or bicyclic ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

Non limiting examples of heterocyclylic moieties include, but are not limited to, the following: pyrazolyl, azepanyl, azabenzimidazole, benzoimidazolyl, benzofuryl, benzofurazanyl, benzopyrazolyl, benzothiazolyl, benzothienyl, benzotriazolyl, benzothiophenyl, benzoxazolyl, carbazolyl, carbolinyl, chromanyl, cinnolinyl, furanyl, imidazolyl, indolinyl, indolyl, indolazinyl, indazolyl, isobenzofuryl, isochromanyl, isoindolyl, isoquinolyl, isothiazolyl, isoxazolyl, naphthpyridinyl, oxadiazolyl, oxazolyl, oxazolinyl, isooxazolinyl, oxetanyl, pyranyl, pyrazinyl, pyrazolidinyl, pyrazolyl, pyridazinyl, pyridopyridinyl, pyridazinyl, pyridinyl, pyrimidyl, pyrrolyl, quinazolinyl, quinolyl, quinoxalinyl, quinoxalinyl, tetrahydropyranyl, tetrahydroisoquinolinyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuryl, dihydrobenzothiophenyl, dihydrobenzoxazolyl, dihydrofuryl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, tetrahydroquinolinyl, tetrahydroisoquinolinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuryl, tetrahydrothienyl, tetrahydroquinolinyl, 2,3-dihydrobenzofuryl, 2,3-dihydrobenzo-1,4-dioxinyl, imidazo(2,1-b)(1,3)thiazole, and benzo-1,3-dioxolyl.

Heteroaromatics form another subset of the heterocycles; i.e., the term “heteroaromatic” (alternatively “heteroaryl”) generally refers to a heterocycle as defined above in which the entire ring system (whether mono- or poly-cyclic) is an aromatic ring system. The term “heteroaromatic ring” refers a 5- or 6-membered monocyclic aromatic ring or a 7- to 12-membered bicyclic which consists of carbon atoms and one or more heteroatoms selected from N, O and S. In the case of substituted heteroaryl rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

“Hydroxyalkyl” refers to an alkyl group as described above in which one or more (in particular 1 to 3) hydrogen atoms have been replaced by hydroxy groups. Examples include CH₂OH, CH₂CHOH and CHOHCH₃.

“Alkyldiyl,” “alkenyldiyl,” “alkynyldiyl”, “cycloalkyldiyl”, “aryldiyl”, “heteroaryldiyl” and “heterocycloalkyldiyl” refer to a divalent radical obtained by the removal of one hydrogen atom from an alkyl, alkenyl, alkynyl, cycloalkyl, aryl, heteroaryl, and heterocycloalkyl group, respectively, each of which is as defined above.

Unless expressly stated to the contrary, all ranges cited herein are inclusive. For example, a heterocycle described as containing from “1 to 4 heteroatoms” means the heterocycle can contain 1, 2, 3 or 4 heteroatoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed.

When any variable (e.g., Rb, etc.) occurs more than one time in any substituent or in Formulas I-II, its definition in each occurrence is independent of its definition at every other occurrence. Also, combinations of substituents and/or variables are permissible only if such combinations result in stable compounds.

In choosing compounds of the present invention, one of ordinary skill in the art will recognize that the various substituents, i.e. R¹, R², R³, etc., are to be chosen in conformity with well-known principles of chemical structure connectivity.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups can be on the same carbon or on different carbons, so long as a stable structure results. The phrase “optionally substituted with one or more substituents” should be taken to be equivalent to the phrase “optionally substituted with at least one substituent” and in such cases one embodiment will have from zero to three substituents.

### Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formulas I and II are shown without a definitive stereochemistry at certain positions. The present invention includes all stereoisomers of Formulas I and II and pharmaceutically acceptable salts and solvates thereof. Unless specifically mentioned otherwise, reference to one isomer applies to any of the possible isomers. Whenever the isomeric composition is unspecified, all possible isomers are included. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I and II may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)— groups (keto forms) may undergo tautomerism to form hydroxy-CH═C(OH)— groups (enol forms). Both keto and enol forms, individually as well as mixtures thereof, are included within the scope of the present invention.

### Salts

Pharmaceutically acceptable salts include both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical stability, flowability, hydro-scopicity and solubility. The term “pharmaceutically acceptable salts” refers to salts prepared from pharmaceutically acceptable non-toxic bases or acids. When the compound of the present invention is acidic, its corresponding salt can be conveniently prepared from inorganic bases or organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from organic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, dicyclohexylamine, lysine, methyl-glucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from inorganic or organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methane-sulfonic, mucic, nitric, pamoic, pantothenic, phosphoric, succinic, sulfuric, tartaric, p-toluene-sulfonic acid and the like. Preferred are citric, hydrobromic, hydrochloric, maleic, phosphoric, sulfuric, and tartaric acids.

### Solvates

The present invention includes within its scope solvates of compounds of Formula I and II. As used herein, the term “solvate” refers to a complex of variable stoichiometry formed by a solute (i.e., a compound of Formula I or II) or a pharmaceutically acceptable salt thereof and a solvent that does not interfere with the biological activity of the solute. Examples of solvents include, but are not limited to water, ethanol, and acetic acid. When the solvent is water, the solvate is known as hydrate; hydrate includes, but is not limited to, hemi-, mono, sesqui-, di- and trihydrates.

### Prodrugs

The present invention includes within its scope the use of prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of Formula I or II, or with a compound which may not be a compound of Formula I or II, but which converts to a compound of Formula I or II in vivo after administration to the patient. Conventional procedures for the selection and preparation of suitable prod rug derivatives are described, for example, in “Design of Prodrugs,” ed. H. Bundgaard, Elsevier, 1985.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I or II. For example, different isotopic forms of hydrogen (H) include protium (1H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I or II can be prepared without undue experimentation by conventional techniques well known to those skilled in the art or by processes analogous to those described in the Schemes and Examples herein using appropriate isotopically-enriched reagents and/or intermediates.

### Utilities

Compounds of the present invention are inhibitors of hypoxia-inducible factor (HIF) prolyl hydroxylases, and as such are useful in the treatment and prevention of diseases and conditions in which HIF modulation is desirable, such as anemia and ischemia. Compounds of the invention can be used in a selective and controlled manner to induce hypoxia-inducible factor stabilization and to rapidly and reversibly stimulate erythropoietin production and secretion. Accordingly, another aspect of the present invention provides a method of treating or preventing a disease or condition in a mammal, the treatment or prevention of which is effected or facilitated by HIF prolyl hydroxylase inhibition, which comprises administering an amount of a compound of Formula I or II that is effective for inhibiting HIF prolyl hydroxylase. This aspect of the present invention further includes the use of a compound of Formula I or II in the manufacture of a medicament for the treatment or prevention of a disease or condition modulated by HIF prolyl hydroxylase.

In one embodiment is a method of enhancing endogenous production of erythropoietin in a mammal which comprises administering to said mammal an amount of a compound of Formula I or II that is effective for enhancing endogenous production of erythropoietin.

Another embodiment is a method of treating anemia in a mammal which comprises administering to said mammal a therapeutically effective amount of a compound of Formulas I or II. “Anemia” includes, but is not limited to, chronic kidney disease anemia, chemotherapy-induced anemia (e.g., anemia resulting from antiviral drug regimens for infectious diseases, such as HIV and hepatitis C virus), anemia of chronic disease, anemia associated with cancer conditions, anemia resulting from radiation treatment for cancer, anemias of chronic immune disorders such as rheumatoid arthritis, inflammatory bowel disease, and lupus, and anemias due to menstruation or of senescence or in other individuals with iron processing deficiencies such as those who are iron-replete but unable to utilize iron properly.

Another embodiment is a method of treating ischemic diseases in a mammal, which comprises administering to said mammal a therapeutically effective amount of a compound of Formulas I or II.

### Combination Therapy

Compounds of Formulas I and II may be used in combination with other drugs that are used in the treatment/prevention/suppression or amelioration of the diseases or conditions for which compounds of Formulas I or II are useful. Such other drugs may be administered, by a route and in an amount commonly used therefor, contemporaneously or sequentially with a compound of Formulas I or II. When a compound of Formulas I or II is used contemporaneously with one or more other drugs, a pharmaceutical composition containing such other drugs in addition to the compound of Formulas Igor II is preferred. Accordingly, the pharmaceutical compositions of the present invention include those that also contain one or more other active ingredients, in addition to a compound of Formulas I or II.

### Route of Administration/Dosage

The compounds of this invention can be administered for the treatment or prevention of afflictions, diseases and illnesses according to the invention by any means that effects contact of the active ingredient compound with the site of action in the body of a warm-blooded animal. For example, administration can be oral, topical, including transdermal, ocular, buccal, intranasal, inhalation, intravaginal, rectal, intracisternal and parenteral. The term “parenteral” as used herein refers to modes of administration which include subcutaneous, intravenous, intramuscular, intraarticular injection or infusion, intrasternal and intraperitoneal. For the purpose of this disclosure, a warm-blooded animal is a member of the animal kingdom possessed of a homeostatic mechanism and includes mammals and birds.

The compounds can be administered by any conventional means available for use in conjunction with pharmaceuticals, either as individual therapeutic agents or in a combination of therapeutic agents. They can be administered alone, but are generally administered with a pharmaceutical carrier selected on the basis of the chosen route of administration and standard pharmaceutical practice.

The dosage administered will be dependent on the age, health and weight of the recipient, the extent of disease, kind of concurrent treatment, if any, frequency of treatment and the nature of the effect desired. Usually, a daily dosage of active ingredient compound will be from about 0.1-2000 milligrams per day. Ordinarily, from 10 to 500 milligrams per day in one or more applications is effective to obtain desired results. These dosages are the effective amounts for the treatment and prevention of afflictions, diseases and illnesses described above, e.g., anemia.

### Pharmaceutical Composition

Another aspect of the present invention provides pharmaceutical compositions which comprises a compound of Formulas I or II and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formulas I or II, additional active ingredient(s), and pharmaceutically acceptable excipients.

The pharmaceutical compositions of the present invention comprise a compound represented by Formulas I or II (or a pharmaceutically acceptable salt or solvate thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The active ingredient can be administered orally in solid dosage forms, such as capsules, tablets, troches, dragées, granules and powders, or in liquid dosage forms, such as elixirs, syrups, emulsions, dispersions, and suspensions. The active ingredient can also be administered parenterally, in sterile liquid dosage forms, such as dispersions, suspensions or solutions. Other dosages forms that can also be used to administer the active ingredient as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Suitable pharmaceutical carriers are described in Remington's Pharmaceutical Sciences, A. Osol, a standard reference text in this field.

For administration by inhalation, the compounds of the present invention may be conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of Formulas I or II in suitable propellants, such as fluorocarbons or hydrocarbons.

For ocular administration, an ophthalmic preparation may be formulated with an appropriate weight percent solution or suspension of the compounds of Formulas I or II in an appropriate ophthalmic vehicle, such that the compound is maintained in contact with the ocular surface for a sufficient time period to allow the compound to penetrate the corneal and internal regions of the eye.

Useful pharmaceutical dosage-forms for administration of the compounds of this invention include, but are not limited to, hard and soft gelatin capsules, tablets, parenteral injectables, and oral suspensions.

A large number of unit capsules are prepared by filling standard two-piece hard gelatin capsules each with 100 milligrams of powdered active ingredient, 150 milligrams of lactose, 50 milligrams of cellulose, and 6 milligrams magnesium stearate.

A mixture of active ingredient in a digestible oil such as soybean oil, cottonseed oil or olive oil is prepared and injected by means of a positive displacement pump into gelatin to form soft gelatin capsules containing 100 milligrams of the active ingredient. The capsules are washed and dried.

A large number of tablets are prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

A parenteral composition suitable for administration by injection is prepared by stirring 1.5% by weight of active ingredient in 10% by volume propylene glycol. The solution is made to volume with water for injection and sterilized.

An aqueous suspension is prepared for oral administration so that each 5 milliliters contain 100 milligrams of finely divided active ingredient, 100 milligrams of sodium carboxymethyl cellulose, 5 milligrams of sodium benzoate, 1.0 grams of sorbitol solution, U.S.P., and 0.025 milliliters of vanillin.

The same dosage forms can generally be used when the compounds of this invention are administered stepwise or in conjunction with another therapeutic agent. When drugs are administered in physical combination, the dosage form and administration route should be selected depending on the compatibility of the combined drugs. Thus the term coadministration is understood to include the administration of the two agents concomitantly or sequentially, or alternatively as a fixed dose combination of the two active components.

Compounds of the invention can be administered as the sole active ingredient or in combination with a second active ingredient, including other active ingredients known to be useful for improving the level of erythropoietin in a patient.

### Abbreviations Used in the Description of the Preparation of the Compounds of the Present Invention

˜ Approximately
AcOH Acetic acid
Ag₂O Silver oxide
AIBN 2,2′-azobis(2-methylpropionitrile)
Aq Aqueous
Bn Benzyl
BnBr Benzylbromide
BnCl Benzylchloride
BnOH Benzylalcohol
Boc₂O or di-tert-butyl dicarbonate
BOC₂O
Brine Saturated aqueous sodium chloride solution
CDI Carbonyl diimidazole
DBU 1,8-diazabicyclo[5.4.0]undec-7-ene
DCC N,N′-dicyclohexylcarbodiimide
DEAD diethylazodicarboxylate
DCM Dichloromethane
DIPEA N,N-diisopropylethylaime
DMA Dimethylacetamide
DMAP 4-N,N-dimethylaminopyridine
DMF N,N-dimethylformamide
DMSO Dimethyl sulfoxide
DPPA Diphenyl phosphoryl azide
EDC or EDCI 1-(3-dimethylaminopropyl)-3-ethylcarbodiimide hydrogen chloride salt
EtOAc or EA Ethyl acetate
Et (et) Ethyl
EtOH Ethanol
Et₂O or ether Diethyl ether
Et₃N triethylamine
g Gram
h or hr Hour
HATU O-(7-Azabenzotriazol-1-yl)-N,N,N′,N′-tetramethyluronium hexafluorophosphate
HCl Hydrochloric acid
HOBt 1-hydroxybenzatriazole
HPLC High-performance liquid chromatography
i-propanol Isopropyl alcohol
i-PrOH or IPA Isopropyl alcohol
K₂CO₃ Potassium carbonate
KOH Potassium hydroxide
LCMS Liquid chromatography mass spectrometry
LiOH Lithium hydroxide
Mg Milligrams
mL Milliliters
mmol Millimole
MeCN Acetonitrile
MeOH Methanol
min Minutes
ms or MS Mass spectrum
μg Microgram(s)
μL Microliters
NaOEt Sodium ethoxide
NaOMe Sodium methoxide
Na₂SO₄ Sodium sulfate
NBS N-bromosuccinimide
NHAc Acetamido
NHCbz Benzyloxycarboxamido
NaOH Sodium hydroxide
NaN₃ Sodium azide
NH₄OH ammonium hydroxide
NMP N-methylpyrrolidone
Pd/C Palladium on carbon
PdCl₂(dppf) [1,1′-Bis(diphenylphosphino)ferrocene]dichloropalladium(II)
Pd(OH)₂ Palladium hydroxide
Pd(PPh₃)₄ Palladium tetrakis(triphenylphosphine)
PE Petroleum ether
PhLi Phenyl lithium
PG Protecting group
Ph Phenyl group
PMB Para-methoxybenzyl
PPTS Pyridinium Para-toluenesulfonate
PPh₃ Triphenyphosphine
Rₜ Retention time
RT or rt Room temperature
SOCl₂ Thionyl chloride
^{t}Bu Tert-butyl
TEA Triethylamine
TFA Trifluoroacetic acid
THF Tetrahydrofuran
TBTU O-(Benzotriazol-1-yl)-N,N,N′,N′-tetramethyluronium tetrafluoroborate
TMS Trimethylsilyl
TMSCl Trimethylsilyl chloride
TsCl Para-toluenesulfonyl chloride

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound in place of multiple substituents which are allowed under the definitions of Formula I or II defined previously.

### General Experimental Comments

Reactions sensitive to moisture or air were performed under nitrogen using anhydrous solvents and reagents. The progress of reactions was determined by either analytical thin layer chromatography (TLC) performed with E. Merck (EMD Millipore, Billerica Mass.) precoated TLC plates, silica gel 60F-254, layer thickness 0.25 mm or liquid chromatography-mass spectrum (LC-MS). Mass analysis was performed on a Waters Micromass® ZQ™ (Waters Corporation, Milford, Mass.) with electrospray ionization in positive ion detection mode. High performance liquid chromatography (HPLC) was conducted on an Agilent 1100 series HPLC on Waters C18 XTerra® 3.5 m 3.0×50 mm column with gradient 10:90-100 v/v CH₃CN/H₂O+v 0.05% TFA over 3.75 min then hold at 100 CH₃CN+v 0.05% TFA for 1.75 min; flow rate 1.0 mL/min, UV wavelength 254 nm). Concentration of solutions was carried out on a rotary evaporator under reduced pressure. Flash chromatography was performed using a Biotage® Flash Chromatography apparatus (Biotage, Charlotte, N.C.) on silica gel (32-63 mM, 60 Å pore size) in pre-packed cartridges. ¹H-NMR spectra were obtained on a 400 or 500 MHz VARIAN® Spectrometer (Varian, Inc. Palo Alto, Calif.) in CDCl₃ or CD₃OD or other solvents as indicated and chemical shifts are reported as δ using the solvent peak as reference and coupling constants are reported in hertz (Hz).

Scheme 1 outlines the general synthetic sequence for compounds of Formula I, which includes compounds of Ia, Ib, Ic and Id. The first step includes the chlorination of 1, followed by amidation with amine 2 to afford amide 3. Upon reaction with benzyl alcohol 4 in the presence of NaH, the benzyloxy-substituted intermediate 5 is obtained. Next, carbonylation and ester hydrolysis gives acid 7, which is followed by amide formation with amino-acid ester 8 to provide 9. Here, L is a carbon linker or multi-carbon linker, or alternatively, L along with the adjacent nitrogen can join together to form a ring system. Debenzylation of 9 gives compounds of general Formula Ia. Alternatively, the ester hydrolysis of 9 affords acid 10. Condensation of the acid 10 with a corresponding alcohol followed by debenzylation result in compounds of general Formula Ia. When both Bn ether and ester are deprotected from compound 9, acid 11 is obtained. Reaction of acid 11 with epoxides give products of Formula Ib. Alternatively, S_{N2} type reaction of acid 11 with chlorides 12 provide products of Formula Ic. Additionally, S_{N2} type reaction of acid 10 with chlorides 12 followed by debenzylation also give compounds of general Formula Ic. Finally, reaction of acid 10 with chlorides 13 (compound 12 where R₄═H) followed by debenzylation give products of Formula Id.

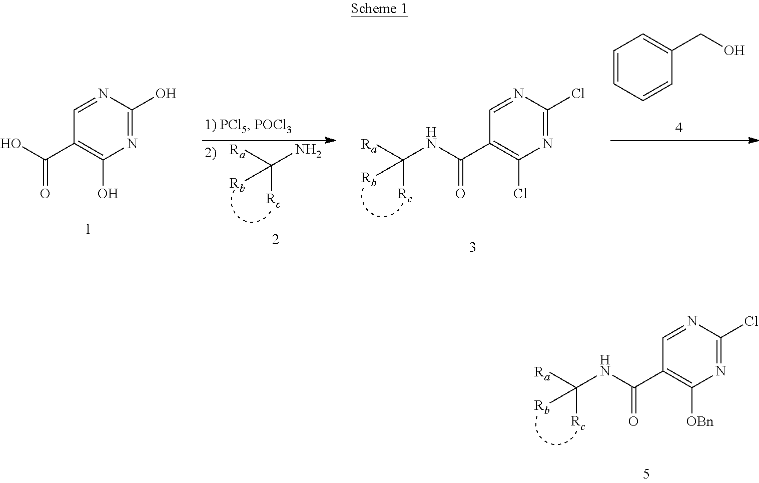

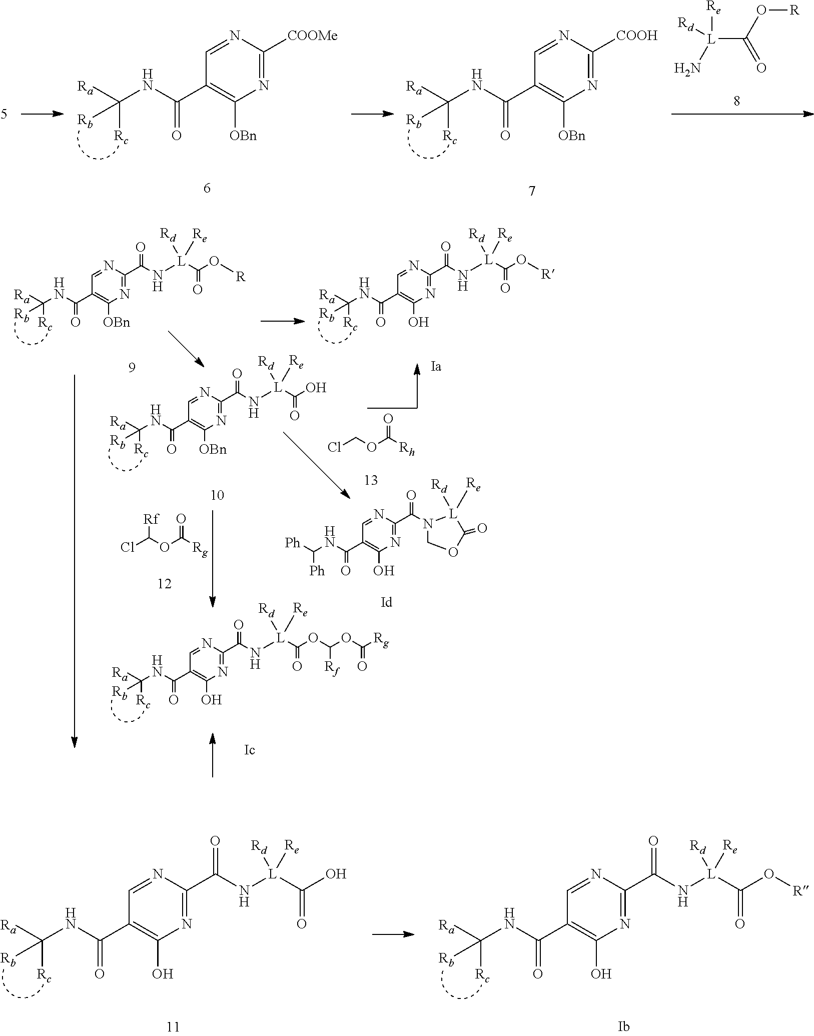

Starting materials useful for the preparation of the compounds in the present invention are known in the art or may be prepared using chemical methodologies known to those skilled in the art.

### Example 1

### Methyl 3-(5-(benzhydrylcarbamoyl)-4-hydroxypyrimidine-2-carboxamido)propanoate

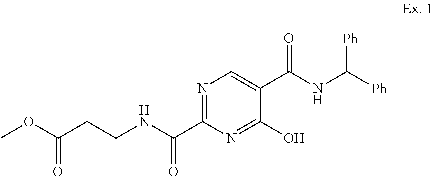 [SMILES: COC(=O)CCNC(=O)c1ncc(C(=O)NC(c2ccccc2)c2ccccc2)c(O)n1]

### Step A N-Benzhydryl-2,4-dichloropyrimidine-5-carboxamide

A 1 L flask was charged with POCl₃ (100 mL), 2,4-dihydroxypyrimidine-5-carboxylic acid (10 g, 0.064 mol) was then added, followed by PCl₅ (14.7 g, 0.071 mol). The mixture was refluxed for 6 hours. After cooling and concentration, the residue was co-evaporated with toluene (100 mL) twice to remove remaining POCl₃. The residue was then dissolved in DCM (100 mL), and the solution was added dropwise to a solution of diphenylmethanamine (12.9 g, 0.07 mol) and TEA (21 g, 0.2 mol) in anhydrous DCM (400 mL) at ˜0° C. After stirring for 30 min at RT, the mixture was washed with water (200 mL) and then dried with sodium sulfate. After removal of a portion of solvent, the crystallized product was collected by suction. The filter cake was taken into EtOAc (400 mL) and the solution was washed with hydrochloric acid (5%, 200 mL), water (200 mL) and brine (100 mL) dried over anhydrous Na₂SO₄ and concentrated to afford the desired product, N-benzhydryl-2,4-dichloropyrimidine-5-carboxamide, as a solid. ¹H NMR (CDCl₃, 400 MHz) δ 8.98 (s, 1H), 7.39-7.29 (m, 10H), 7.23 (d, J=7.3 Hz, 1H), 6.41 (d, J=7.3 Hz, 1H). LC/MS (m/z): 358 (M+H)⁺.

### Step B N-Benzhydryl-4-(benzyloxy)-2-chloropyrimidine-5-carboxamide

To a solution of benzyl alcohol (2.5 ml, 23.9 mmol) in anhydrous THF (100 ml) was slowly added NaH (1.0 g, 26.0 mmol) at 0° C. and the reaction mixture was allowed to stir at room temperature for 0.5 h. The resulting suspension was then slowly added to an ice-salt-cooled solution of N-benzhydryl-2,4-dichloropyrimidine-5-carboxamide (10.0 g, 21.7 mmol) in THF (150 mL) through an addition funnel, so as to keep the reaction temperature under 0° C. After the addition, the reaction mixture was stirred at 0° C. for 1 hour, when LCMS showed the reaction completed. The reaction mixture was acidified with 5% HCl at ˜0° C. to pH 6-7 and then extracted with EtOAc (200 mL). The organic phase was washed with water and brine (200 mL each), dried over Na₂SO₄ and concentrated under vacuum to afford a crude product. The crude product was purified by recrystallization from EtOAc/Petroleum ether to afford N-benzhydryl-4-(benzyloxy)-2-chloropyrimidine-5-carboxamide as a solid. ¹H NMR (CDCl₃, 400 MHz) δ 9.17 (s, 1H), 8.11 (d, J=8.0 Hz, 1H), 7.39-7.33 (m, 5H), 7.22-7.19 (m, 6H), 7.05-7.03 (m, 4H), 6.34 (d, J=8.0 Hz, 1H), 5.53 (s, 2H). LC/MS (m/z): 430 (M+H)⁺.

### Step C Methyl 5-(benzhydrylcarbamoyl)-4-(benzyloxy)pyrimidine-2-carboxylate

To a 2 L stainless steel autoclave were added N-benzhydryl-4-(benzyloxy)-2-chloropyrimidine-5-carboxamide (20.0 g, 46.6 mmol), Pd(dppf)Cl₂ (3.4 g, 4.66 mmol), NaOAc (11.6 g, 139.8 mmol) and MeOH (800 mL). The air in the autoclave was replaced with carbon monoxide and the pressure was adjusted to 3.6 MPa. The reaction mixture was then stirred at 70° C. for 3 hours. After cooling, the filtrate was concentrated under vacuum. The residue was purified by column chromatography on silica gel eluting with Petroleum ether/EtOAc (3:1 to 1:1) to afford methyl 5-(benzhydrylcarbamoyl)-4-(benzyloxy)pyrimidine-2-carboxylate as a solid. ¹H NMR (CDCl₃, 400 MHz) δ 9.42 (s, 1H), 8.29 (d, J=7.9 Hz, 1H), 7.40-7.33 (m, 5H), 7.23-7.21 (m, 6H), 7.07-7.04 (m, 4H), 6.35 (d, J=7.9 Hz, 1H), 5.64 (s, 2H), 4.05 (s, 3H). LC/MS (m/z): 454 (M+H)⁺.

### Step D 5-(Benzhydrylcarbamoyl)-4-(benzyloxy)pyrimidine-2-carboxylic acid

To a solution of methyl 5-(benzhydrylcarbamoyl)-4-(benzyloxy)pyrimidine-2-carboxylate (10.0 g, 22.8 mmol) in THF (350 mL) was added aq. NaOH (5%, 22 mL, 27.3 mmol) over a 50 min period. After the addition was done, the reaction mixture was allowed to stir at RT for 30 min until TLC showed the reaction was completed. The mixture was acidified with aq. HCl (5%) to pH of about 3-4, diluted with DCM (500 mL) and then washed with brine (300 mL). The organic layer was dried over Na₂SO₄ and concentrated under vacuum to about 50 mL. To the residue was added petroleum ether (200 mL). The formed precipitate was collected by filtration to afford 5-(benzhydrylcarbamoyl)-4-(benzyloxy)pyrimidine-2-carboxylic acid as a solid. ¹H NMR (CDCl₃, 400 MHz) δ 9.34 (s, 1H), 8.21 (d, J=7.9 Hz, 1H), 7.36-7.29 (m, 5H), 7.19-7.17 (m, 6H), 7.00-6.98 (m, 4H), 6.29 (d, J=7.9 Hz, 1H), 5.66 (s, 2H). LC/MS (m/z): 440 (M+H)⁺.

### Step E Methyl 3-(5-(benzhydrylcarbamoyl)-4-(benzyloxy)pyrimidine-2-carboxamido)propanoate

To a 20 mL single-neck flask were added 5-(benzhydrylcarbamoyl)-4-(benzyloxy) pyrimidine-2-carboxylic acid (100 mg, 0.22 mmol), DCM (3 mL), TEA (90 mg, 0.9 mmol) and methyl 3-aminopropanoate hydrochloride (63 mg, 0.45 mmol). After stirring for 10 min, the solution was cooled to 0° C. with salt-ice. To the chilled solution was added HATU (170 mg, 0.45 mmol). The reaction solution was stirred for 10 h at room temperature until LCMS showed the reaction was completed. The reaction solution was diluted with EtOAc (20 mL), washed with H₂O (20 mL) and brine (20 mL), dried over Na₂SO₄ and concentrated under reduced pressure to afford crude product. The crude product was then purified by column chromatography on silica gel (eluted with ethyl acetate/Petroleum ether 1:1 vol/vol.) to give the desired product as a solid. ¹H NMR (CDCl₃, 400 MHz) δ 9.30 (s, 1H), 8.46 (m, 1H), 8.23 (d, J=8.2 Hz, 1H), 7.34-7.29 (m, 5H), 7.17-7.15 (m, 6H), 7.07-7.05 (m, 4H), 6.35 (d, J=8.2 Hz, 1H), 5.67 (s, 2H), 3.78 (q, J=6.0 Hz, 2H), 3.72 (s, 3H), 2.69 (t, J=6.0 Hz, 2H). LC/MS (m/z): 525 (M+H)⁺.

### Step F Methyl 3-(5-(benzhydrylcarbamoyl)-4-hydroxypyrimidine-2-carboxamido)propanoate (Ex. 1)

To a solution of methyl 3-(5-(benzhydrylcarbamoyl)-4-(benzyloxy)pyrimidine-2-carboxamido) propanoate (80 mg, 0.15 mmol) in EtOAc (10 mL) was added Pd/C (wet/10%, 10 mg). The mixture was stirred under hydrogenation atmosphere for 3 h. When TLC showed the reaction completed, the mixture was filtered through a pad of CELITE and the filtration was concentrated under vacuum to give desired compound, Ex. 1, as a solid. ¹H NMR (DMSO-d₆, 400 MHz) δ 11.82 (d, J=8.2 Hz, 1H), 8.68-8.65 (m, 1H), 8.54 (s, 1H), 7.33-7.27 (m, 8H), 7.22-7.20 (m, 2H), 6.23 (d, J=8.4 Hz, 1H), 3.58 (s, 3H), 3.47-3.42 (m, 2H), 2.57-2.54 (m, 2H). LC/MS (m/z): 457 (M+Na)⁺. Human HIF-PHD2 IC₅₀: 14.3 nM.

Examples 2-5 as shown in Table 1 were prepared utilizing synthesis methods analogous to those described in Example 1 and using the appropriate starting materials.

TABLE 1

| | | | MS m/z |
|---|---|---|---|
| | | | (M + 1)⁺ and |
| | | | human HIF- |
| Example | Name | Structure | PHD2 IC₅₀ |
| Ex. 2 | 2-morpholinoethyl 3-(5- (benzhydrylcarbamoyl)- 4- hydroxypyrimidine- 2-carboxamido) propanoate | 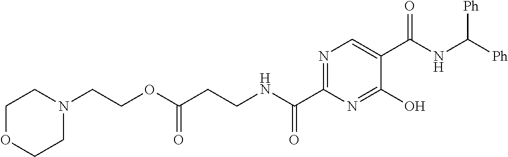 [SMILES: O=C(CCNC(=O)c1ncc(C(=O)NC(c2ccccc2)c2ccccc2)c(O)n1)OCCN1CCOCC1] | (M + 1)⁺ 534 IC₅₀ 24.9 nM |
| Ex. 3 | ethyl 1-((5- (benzhydrylcarbamoyl)- 4- hydroxypyrimidine- 2-carboxamido) methyl)cyclo- propanecarboxylate | 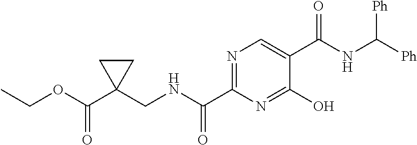 [SMILES: CCOC(=O)C1(CNC(=O)c2ncc(C(=O)NC(c3ccccc3)c3ccccc3)c(O)n2)CC1] | (M + 1)⁺ 475 IC₅₀ 35.6 nM |
| Ex. 4 | ethyl 3-(5- (benzhydrylcarbamoyl)- 4- hydroxypyrimidine-2- carboxamido)-2- hydroxy-2- methylpropanoate | 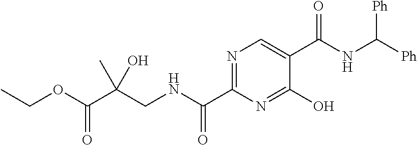 [SMILES: CCOC(=O)C(C)(O)CNC(=O)c1ncc(C(=O)NC(c2ccccc2)c2ccccc2)c(O)n1] | (M + 1)⁺ 479 IC₅₀ 871.3 nM |
| Ex. 5 | (R)-ethyl 3-(5- (benzhydrylcarbamoyl)- 4- hydroxyprimidine-2- carboxamido)-2- hydroxypropanoate | 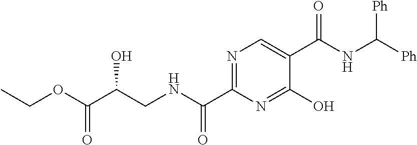 [SMILES: CCOC(=O)[C@H](O)CNC(=O)c1ncc(C(=O)NC(c2ccccc2)c2ccccc2)c(O)n1] | (M + 1)⁺ 465 IC₅₀ 46.4 nM |

### Example 6

### Isopropyl 3-(5-(benzhydrylcarbamoyl)-4-hydroxypyrimidine-2-carboxamido)propanoate

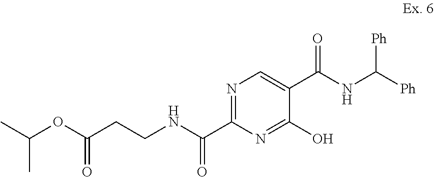 [SMILES: CC(C)OC(=O)CCNC(=O)c1ncc(C(=O)NC(c2ccccc2)c2ccccc2)c(O)n1]

A round bottom flask equipped with a stirring bar was charged with propan-2-ol (8 mL). NaH (55 mg, 1.39 mmol) was then added. The reaction mixture was stirred at room temperature for 0.5 hours and methyl 3-(5-(benzhydrylcarbamoyl)-4-hydroxypyrimidine-2-carboxamido)propanoate (100 mg, 0.23 mmol) was added and further stirred for 5 min at room temperature. When TLC showed the reaction completed, the reaction mixture was slowly poured to aq. HCl (1M, 20 mL) at 0° C. and the resulting suspension was filtered to afford a crude product. The crude product was further purified by silica gel chromatography eluting with EtOAc to afford isopropyl 3-(5-(benzhydrylcarbamoyl)-4-hydroxypyrimidine-2-carboxamido)propanoate. ¹H NMR (CDCl₃, 400 MHz) δ 11.11 (s, 1H), 9.90 (d, J=8.0 Hz, 1H), 8.97 (s, 1H), 8.40-8.34 (m, 1H), 7.33-7.28 (m, 8H), 7.25-7.20 (m, 2H), 6.43 (d, J=8.0 Hz, 1H), 5.09-5.03 (m, 1H), 3.71 (q, J=5.8 Hz, 2H), 2.61 (t, J=5.8 Hz, 2H), 1.25 (d, J=6.2 Hz, 6H). LC/MS (m/z): 485 (M+Na)⁺. Human HIF-PHD2 IC₅₀: 15.9 nM.

### Example 7

### Acetoxymethyl 3-(5-(benzhydrylcarbamoyl)-4-hydroxypyrimidine-2-carboxamido)propanoate

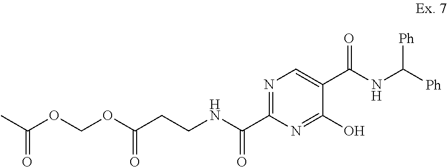 [SMILES: CC(=O)OCOC(=O)CCNC(=O)c1ncc(C(=O)NC(c2ccccc2)c2ccccc2)c(O)n1]

### Step A tert-Butyl 3-(5-(benzhydrylcarbamoyl)-4-(benzyloxy)pyrimidine-2-carboxamido)propanoate

To a mixture of 5-(benzhydrylcarbamoyl)-4-(benzyloxy)pyrimidine-2-carboxylic acid (5.0 g, 11.4 mmol), tert-butyl 3-aminopropanoate (4.2 g, 22.8 mmol) and TEA (10.0 mL, 45.6 mmol) in DCM (1000 ml) was added HATU (5.2 g, 13.7 mmol). The reaction mixture was stirred at RT for 16 hours. After concentration under vacuum, the crude product was purified by silica gel chromatography eluting with PE/EA (2:3) to afford tert-butyl 3-(5-(benzhydrylcarbamoyl)-4-(benzyloxy)pyrimidine-2-carboxamido)propanoate. ¹H NMR (CDCl₃, 400 MHz) δ 9.37 (s, 1H), 8.51 (m, 1H), 8.29 (d, J=8.0 Hz, 1H), 7.40-7.33 (m, 5H), 7.23-7.21 (m, 6H), 7.07-7.04 (m, 4H), 6.32 (d, J=8.0 Hz, 1H), 5.66 (s, 2H), 3.62 (q, J=6.0 Hz, 2H), 2.59 (t, J=6.0 Hz, 2H), 1.46 (s, 9H). LC/MS (m/z): 567 (M+H)⁺.

### Step B tert-Butyl 3-(5-(benzhydrylcarbamoyl)-4-hydroxypyrimidine-2-carboxamido)propanoate

To a solution of tert-butyl 3-(5-(benzhydrylcarbamoyl)-4-(benzyloxy)pyrimidine-2-carboxamido)propanoate (4.0 g, 7.06 mmol) in EtOAc (1000 mL) was added Pd/C (10%, 0.4 g). The mixture was stirred under H₂ atmosphere for 2 hours until LCMS showed starting material was consumed. After filtration through a pad of CELITE, the filtrate was concentrated under vacuum to afford the desired tert-butyl 3-(5-(benzhydrylcarbamoyl)-4-(benzyloxy)pyrimidine-2-carboxamido)propanoate. ¹H NMR (DMSO-d₆, 400 MHz) δ 9.18 (s, 1H), 7.35-7.28 (m, 9H), 7.26-7.22 (m, 2H), 6.24 (d, J=7.9 Hz, 1H), 3.47-3.42 (m, 2H), 2.51-2.47 (m, 2H), 1.36 (s, 9H). LC/MS (m/z): 477 (M+H)⁺.

### Step C 3-(5-(Benzhydrylcarbamoyl)-4-hydroxypyrimidine-2-carboxamido)propanoic acid

A solution of tert-butyl 3-(5-(benzhydrylcarbamoyl)-4-hydroxypyrimidine-2-carboxamido) propanoate (3.0 g, 6.3 mmol) in HCl (4M in dioxane) was stirred at RT for 6 hours. The reaction mixture was then concentrated under vacuum to about 50 mL. To the residue was added petroleum ether (150 mL), and the precipitate was collected by filtration to afford the desired product, 3-(5-(benzhydrylcarbamoyl)-4-hydroxypyrimidine-2-carboxamido) propanoic acid. ¹H NMR (400 MHz, DMSO-d6) δ 10.39 (s, 1H), 9.18 (m, 1H), 8.47 (s, 1H), 7.35-7.29 (m, 8H), 7.26-7.22 (m, 2H), 6.25 (d, J=7.9 Hz, 1H), 3.46 (q, J=6.9 Hz, 2H), 2.52 (t, J=6.9 Hz, 2H). LC/MS (m/z): 421 (M+H)⁺.

### Step D Acetoxymethyl 3-(5-(benzhydrylcarbamoyl)-4-hydroxypyrimidine-2-carboxamido)propanoate

To a solution of 3-(5-(benzhydrylcarbamoyl)-4-hydroxypyrimidine-2-carboxamido)propanoic acid (100 mg, 0.238 mmol) in DMF (1.5 mL) were added chloromethyl acetate (39 mg, 0.36 mmol) and K₂CO₃ (65.6 mg, 0.476 mmol). The resulting mixture was stirred at 25° C. for 16 h, when LCMS showed the reaction completed. The reaction mixture was filtered and then concentrated. The residue was purified by Prep-HPLC (Column: Gemini 150*21.5 mm*5 um (Phenomenex, Inc. Torrance Calif., USA); Mobile phase: from 28% MeCN in water (NH₄HCO₃, 0.05 mol/L) to 56% MeCN in water (NH₄HCO₃, 0.05 mol/L); Wavelength: 220 nm) to give the acetoxymethyl 3-(5-(benzhydrylcarbamoyl)-4-hydroxypyrimidine-2-carboxamido) propanoate, Ex. 7. ¹H NMR (CDCl₃, 400 MHz): δ 9.86 (d, J=8.0 Hz, 1H), 8.94 (s, 1H), 8.19 (t, J=6.0 Hz, 1H), 7.35-7.28 (m, 10H), 6.44 (d, J=8.0 Hz, 1H), 5.76 (s, 2H), 3.76 (q, J=6.4 Hz, 2H), 2.72 (t, J=6.4, 2H), 2.10 (s, 3H). LC/MS (m/z): 493 (M+H)⁺. Human HIF-PHD2 IC₅₀: 3.11 nM.

Examples 8-12 in Table 2 were prepared using analogous synthesis procedures as those described in Example 7 in conjunction with the appropriate starting materials.

TABLE 2

| | | | MS m/z |
|---|---|---|---|
| | | | (M + 1)⁺ and |
| | | | human HIF- |
| Example | Name | Structure | PHD2 IC₅₀ |
| Ex. 8 | ((isopropoxycarbonyl)oxy) methyl 3-(5- (benzhydrylcarbamoyl)-4- hydroxypyrimidine-2- carboxamido)propanoate | 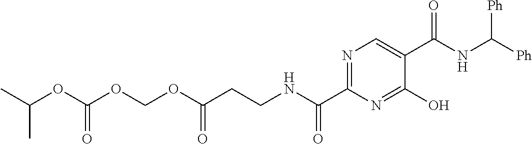 [SMILES: CC(C)OC(=O)OCOC(=O)CCNC(=O)c1ncc(C(=O)NC(c2ccccc2)c2ccccc2)c(O)n1] | (M + 1)⁺ 537 IC₅₀ 2.81 nM |
| Ex. 9 | 1-((ethoxycarbonyl)oxy) ethyl 3-(5- (benzhydrylcarbamoyl)-4- hydroxypyrimidine-2- carboxamido)propanoate | 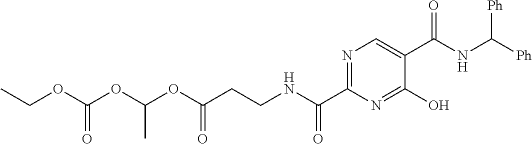 [SMILES: CCOC(=O)OC(C)OC(=O)CCNC(=O)c1ncc(C(=O)NC(c2ccccc2)c2ccccc2)c(O)n1] | (M + 1)⁺ 537 IC₅₀ 6.67 nM |
| Ex. 10 | 1-((isopropoxycarbonyl) oxy)ethyl 3-(5- (benzhydrylcarbamoyl)-4- hydroxypyrimidine-2- carboxamido)propanoate | 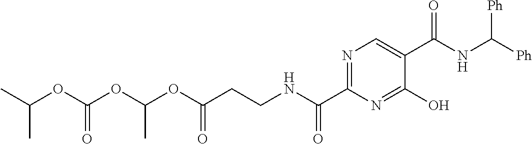 [SMILES: CC(C)OC(=O)OC(C)OC(=O)CCNC(=O)c1ncc(C(=O)NC(c2ccccc2)c2ccccc2)c(O)n1] | (M + 1)⁺ 551 IC₅₀ 37.2 nM |
| Ex. 11 | 1-(((cyclohexyloxy) carbonyl)oxy)ethyl 3-(5- (benzhydrylcarbamoyl)-4- hydroxyprimidine-2- carboxamido)propanoate | 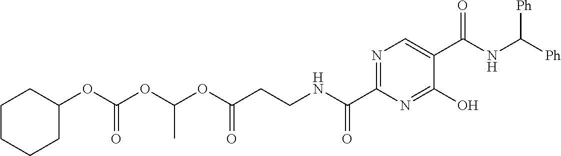 [SMILES: CC(OC(=O)CCNC(=O)c1ncc(C(=O)NC(c2ccccc2)c2ccccc2)c(O)n1)OC(=O)OC1CCCCC1] | (M + 1)⁺ 591 IC₅₀ 42.1 nM |
| Ex. 12 | ((ethoxycarbonyl)oxy) methyl 3-(5- (benzhydrylcarbamoyl)-4- hydroxypyrimidine-2- carboxamido)propanonate | 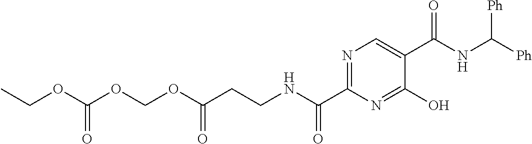 [SMILES: CCOC(=O)OCOC(=O)CCNC(=O)c1ncc(C(=O)NC(c2ccccc2)c2ccccc2)c(O)n1] | (M + 1)⁺ 523 IC₅₀ 2.46 nM |

### Example 13

### 2-(Dimethylamino)-2-oxoethyl 3-(5-(benzhydrylcarbamoyl)-4-hydroxypyrimidine-2-carboxamido)propanoate

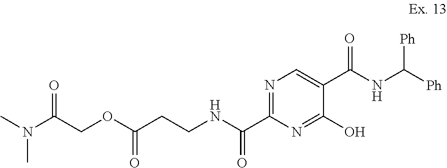 [SMILES: CN(C)C(=O)COC(=O)CCNC(=O)c1ncc(C(=O)NC(c2ccccc2)c2ccccc2)c(O)n1]

### Step A 2-(Dimethylamino)-2-oxoethyl 3-(5-(benzhydrylcarbamoyl)-4-(benzyloxy)pyrimidine-2-carboxamido)propanoate

To a solution of methyl 3-(5-(benzhydrylcarbamoyl)-4-(benzyloxy)pyrimidine-2-carboxamido) propanoate (0.5 g, 0.95 mmol) in THF (50 mL) was added NaOH (0.046 g, 1.14 mmol in 10 mL H₂O) dropwise over 30 min. After the addition, the reaction mixture was stirred at room temperature for 45 min, until TLC showed all of starting material was consumed. The mixture was acidified to pH 3-4 with dilute aq. HCl (5%), diluted with brine (20 mL), and then extracted with DCM (100 mL). The organic layer was dried over Na₂SO₄ and concentrated under vacuum to remove most of solvent. The residue allowed to stand still for 1 h, and the precipitate was collected by filtration to afford desired 3-(5-(benzhydrylcarbamoyl)-4-(benzyloxy)pyrimidine-2-carboxamido)propanoic acid, which was used in the next step directly. LC/MS (m/z): 511 (M+H)⁺.

### Step B 2-(Dimethylamino)-2-oxoethyl 3-(5-(benzhydrylcarbamoyl)-4-(benzyloxy)pyrimidine-2-carboxamido)propanoate

To a solution of 3-(5-(benzhydrylcarbamoyl)-4-(benzyloxy)pyrimidine-2-carboxamido) propanoic acid (200 mg, 0.39 mmol) and 2-chloro-N,N-dimethylacetamide (143 mg, 1.18 mmol) in DMF (10 mL) was added K₂CO₃ (160 mg, 1.18 mmol). The mixture was stirred at room temperature for 16 hours. When LCMS showed the reaction completed, the reaction mixture was poured into H₂O (100 mL) and the precipitate was collected by filtration to afford the crude product. The crude product was recrystallized from EtOAc to afford 2-(dimethylamino)-2-oxoethyl 3-(5-(benzhydrylcarbamoyl)-4-(benzyloxy)pyrimidine-2-carboxamido)propanoate. ¹H NMR (methanol-d₄, 400 MHz) δ 9.04 (s, 1H), 7.51-7.45 (m, 2H), 7.43-7.34 (m, 3H), 7.24-7.18 (m, 6H), 7.13-7.07 (m, 4H), 6.26 (s, 1H), 5.67 (s, 2H), 3.78 (t, J=6.4 Hz, 2H), 2.95 (s, 3H), 2.84 (s, 3H), 2.78 (t, J=6.4 Hz, 2H). LC/MS (m/z): 596 (M+H)⁺.

### Step C 2-(Dimethylamino)-2-oxoethyl 3-(5-(benzhydrylcarbamoyl)-4-hydroxypyrimidine-2-carboxamido)propanoate

To a solution of 2-(dimethylamino)-2-oxoethyl 3-(5-(benzhydrylcarbamoyl)-4-(benzyloxy)pyrimidine-2-carboxamido)propanoate (110 mg, 0.185 mmol) in EtOAc (150 mL) was added Pd/C (10%, 50 mg). The mixture was stirred under a hydrogenation atmosphere for 2 h. When TLC showed the reaction completed, the mixture was filtered. The filtrate was concentrated under vacuum to afford 2-(dimethylamino)-2-oxoethyl 3-(5-(benzhydrylcarbamoyl)-4-hydroxypyrimidine-2-carboxamido)propanoate, Ex. 13. ¹H NMR (CDCl₃, 400 MHz) δ 9.93 (s, 1H), 8.95 (s, 1H), 8.87 (s, 1H), 7.35-7.28 (m, 8H), 7.25-7.22 (m, 4H), 6.42 (d, J=8.2 Hz, 1H), 4.79 (s, 2H), 3.83 (q, J=5.9 Hz, 2H), 2.96 (s, 6H), 2.73 (t, J=5.7 Hz, 2H). LC/MS (m/z): 528 (M+Na)⁺. Human HIF-PHD2 IC₅₀: 158.8 nM.

### Example 14

### N-Benzhydryl-4-hydroxy-2-(6-oxo-1,3-oxazinane-3-carbonyl)pyrimidine-5-carboxamide

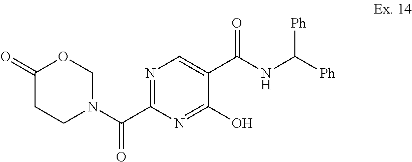 [SMILES: O=C1CCN(C(=O)c2ncc(C(=O)NC(c3ccccc3)c3ccccc3)c(O)n2)CO1]

### Step A N-benzhydryl-4-(benzyloxy)-2-(6-oxo-1,3-oxazinane-3-carbonyl)pyrimidine-5-carboxamide

To a solution of 3-(5-(benzhydrylcarbamoyl)-4-(benzyloxy)pyrimidine-2-carboxamido) propanoic acid (200 mg, 0.39 mmol) in DMF (2 mL) were added chloromethyl pivalate (175 mg, 1.15 mmol) and K₂CO₃ (107.6 mg, 0.78 mmol). The resulting mixture was stirred at 25° C. for 16 h. LCMS showed the reaction was complete. The reaction mixture was diluted with EtOAC (10 mL) and filtered. The filtrate was concentrated. The residue was purified by silica gel chromatography (petroleum ether in EtOAc=20%˜50%) to give N-benzhydryl-4-(benzyloxy)-2-(6-oxo-1,3-oxazinane-3-carbonyl)pyrimidine-5-carboxamide. ¹H NMR (CDCl₃, 400 MHz): δ 9.38 (s, 1H), 8.25 (d, J=8.0 Hz, 1H), 7.41-7.36 (m, 5H), 7.26-7.23 (m, 6H), 7.23-7.06 (m, 4H), 6.36 (d, J=8.0 Hz, 1H), 5.80 (s, 0.5H), 5.60 (s, 2H), 5.55 (s, 1.5H), 4.06 (d, J=7.2 Hz, 1.5H), 3.79 (d, J=7.2 Hz, 0.5H), 2.97 (d, J=7.2 Hz, 1.5H), 2.84 (d, J=7.2 Hz, 0.5H). LC/MS (m/z): 523 (M+H)⁺.

### Step B N-benzhydryl-4-hydroxy-2-(6-oxo-1,3-oxazinane-3-carbonyl)pyrimidine-5-carboxamide

To a solution of N-benzhydryl-4-(benzyloxy)-2-(6-oxo-1,3-oxazinane-3-carbonyl)pyrimidine-5-carboxamide (80 mg, 0.15 mmol) in EtOAc (5 mL) was added Pd/C (10%, 10 mg). The mixture was stirred under hydrogen (balloon) at room temperature for 5 h. The reaction mixture was filtered and the filtrate was concentrated to afford N-benzhydryl-4-hydroxy-2-(6-oxo-1,3-oxazinane-3-carbonyl)pyrimidine-5-carboxamide, Ex. 14. ¹H NMR (CDCl₃, 400 MHz): δ 11.25 (brs, 1H), 10.09 (br s, 1H), 8.75 (s, 1H), 7.52-7.03 (m, 10H), 6.30 (d, J=7.53 Hz, 1H), 6.02 (s, 1.4H), 5.67 (s, 0.6H), 4.23 (t, J=7.28 Hz, 0.6H), 3.88 (t, J=7.28 Hz, 1.4H), 2.94 (t, J=7.28 Hz, 1.4H), 2.88 (t, J=7.28 Hz, 0.6H). LC/MS (m/z): 433 (M+H)+. Human HIF-PHD2 IC₅₀: 11.2 nM.

### Example 15

### 2,3-Dihydro-1H-inden-5-yl 2-(5-(benzhydrylcarbamoyl)-4-hydroxypyrimidine-2-carboxamido)acetate

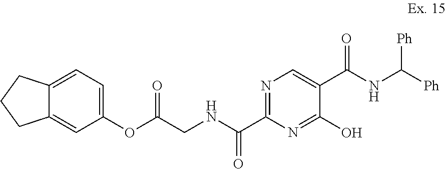 [SMILES: O=C(CNC(=O)c1ncc(C(=O)NC(c2ccccc2)c2ccccc2)c(O)n1)Oc1ccc2c(c1)CCC2]

### Step A Methyl 2-(5-(benzhydrylcarbamoyl)-4-(benzyloxy)pyrimidine-2-carboxamido)acetate

To a solution of 5-(benzhydrylcarbamoyl)-4-(benzyloxy)pyrimidine-2-carboxylic acid (6.5 g, 14.8 mmol) and methyl 2-aminoacetate hydrochloride (2.79 g, 22.2 mmol) in DMF (70 mL) were added HATU (11.25 g, 29.6 mmol) and Et₃N (4.48 g, 44.4 mmol). The mixture was stirred for 2 h at room temperature. LCMS showed the reaction was complete. The reaction mixture was diluted with water (200 mL) and extracted with EtOAc (100 mL×3). The combined organic layers were dried over Na₂SO₄ and concentrated. The residue was purified by CombiFlash® (MeOH in DCM=0%˜3%) to give desired product. ¹H NMR (CDCl₃, 400 MHz): δ 9.39 (s, 1H), 8.41 (br s, 1H), 8.29 (d, J=7.9 Hz, 1H), 7.39-7.05 (m, 15H), 6.35 (d, J=7.9 Hz, 1H), 5.67 (s, 2H), 4.30 (d, J=5.5 Hz, 2H), 3.81 (s, 3H). LC/MS (m/z): 511 (M+H)⁺.

### Step B 2-(5-(Benzhydrylcarbamoyl)-4-(benzyloxy)pyrimidine-2-carboxamido)acetic acid

To a solution of methyl 2-(5-(benzhydrylcarbamoyl)-4-(benzyloxy)pyrimidine-2-carboxamido) acetate (2.1 g, 4.11 mol) in THF (400 mL) and H₂O (80 mL) was added IN NaOH (4.52 mL, 4.52 mmol) within 15 min at 0° C.˜5° C. After addition, the mixture was stirred for 5 min. TLC (DCM:MeOH=15:1) showed the reaction was complete. The mixture was diluted with water (500 mL) and extracted with EtOAc (500 mL×2). The aqueous layer was acidified to pH 3˜4 with IN HCl and then extracted with EtOAc (500 mL×3). The combined organic layers were concentrated under vacuum to give desired product. ¹H NMR (DMSO-d₆, 400 MHz): δ 9.19 (t, J=6.0 Hz, 1H), 9.07 (d, J=8.2 Hz, 1H), 8.86 (s, 1H), 7.52-7.20 (m, 15H), 6.22 (d, J=8.2 Hz, 1H), 5.60 (s, 2H), 3.97 (d, J=6.0 Hz, 2H). LC/MS (m/z): 497 (M+H)⁺.

### Step C 2,3-Dihydro-1H-inden-5-yl 2-(5-(benzhydrylcarbamoyl)-4-(benzyloxy)pyrimidine-2-carboxamido)acetate

To a solution of 2-(5-(benzhydrylcarbamoyl)-4-(benzyloxy)pyrimidine-2-carboxamido)acetic acid (0.25 g, 0.504 mol) in DCM (5 mL) were added 2,3-dihydro-1H-inden-5-ol (88 mg, 0.66 mmol), DCC (136.2 mg, 0.66 mmol) and 4-DMAP (6.1 mg, 0.05 mmol). The mixture was stirred overnight at 25° C. LCMS showed the reaction was complete. The mixture was diluted with water (20 mL) and extracted with EtOAc (20 mL×3). The combined organic layers were concentrated. The residue was purified by prep. TLC (eluted with Petroleum ether/EtOAc 1: vol.: vol.) to give 2,3-dihydro-1H-inden-5-yl 2-(5-(benzhydrylcarbamoyl)-4-(benzyloxy)pyrimidine-2-carboxamido)acetate. ¹H NMR (DMSO-d₆, 400 MHz) δ 9.47 (t, J=5.2 Hz, 1H), 9.08 (d, J=7.9 Hz, 1H), 8.87 (s, 1H), 7.53-7.49 (m, 2H), 7.40-7.36 (m, 3H), 7.23-7.20 (m, 11H), 6.96 (s, 1H), 6.84 (d, J=7.1 Hz, 1H), 6.22 (d, J=8.2 Hz, 1H), 5.61 (s, 2H), 4.31 (d, J=5.7 Hz, 2H), 2.85-2.79 (m, 4H), 2.04-1.99 (m, 2H). LC/MS (m/z): 613 (M+H)⁺.

### Step D 2,3-Dihydro-1H-inden-5-yl 2-(5-(benzhydrylcarbamoyl)-4-hydroxypyrimidine-2-carboxamido)acetate

To a solution of 2,3-dihydro-1H-inden-5-yl 2-(5-(benzhydrylcarbamoyl)-4-(benzyloxy) pyrimidine-2-carboxamido)acetate (150 mg, 0.24 mmol) in EtOAc (20 mL) was added Pd—C(wet, 10%, 15 mg). The reaction mixture was stirred under a hydrogen atmosphere for 3 hours. When LCMS showed that the reaction was completed, the reaction mixture was filtered through a pad of CELITE and the filtrate was concentrated under reduced pressure to afford 2,3-dihydro-1H-inden-5-yl 2-(5-(benzhydrylcarbamoyl)-4-hydroxypyrimidine-2-carboxamido)acetate, Ex. 15. ¹H NMR (DMSO-d6, 400 MHz) δ 13.71 (brs, 1H), 10.50 (brs, 1H), 9.63 (s, 1H), 8.51 (brs, 1H), 7.38-7.25 (m, 11H), 6.97 (s, 1H), 6.87 (d, J=8.0 Hz, 1H), 6.27 (d, J=8.0 Hz, 1H), 4.29 (d, J=6.0 Hz, 2H), 2.84 (t, J=7.6 Hz, 4H), 2.06-2.02 (m, 2H). LC/MS (m/z): 545 (M+Na)⁺. Human HIF-PHD2 IC₅₀: 11.2 nM.

Example 16 in Table 3 was prepared using analogous synthesis procedures as those described in Example 15 in conjunction with the appropriate starting materials.

TABLE 3

| | | | MS m/z |
|---|---|---|---|
| | | | (M + 1)⁺ and |
| | | | human HIF- |
| Example | Name | Structure | PHD2 IC₅₀ |
| Ex. 16 | 2-(dimethylamino)-2- oxoethyl 2-(5- (benzhydrylcarbamoyl)- 4-hydroxypyrimidine-2- carboxamido)acetate | 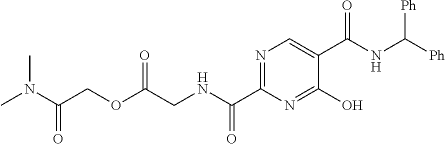 [SMILES: CN(C)C(=O)COC(=O)CNC(=O)c1ncc(C(=O)NC(c2ccccc2)c2ccccc2)c(O)n1] | (M + 1)⁺ 492 IC₅₀ 104.8 nM |

### Example 17

### N-Benzhydryl-4-hydroxy-2-(5-oxooxazolidine-3-carbonyl)pyrimidine-5-carboxamide

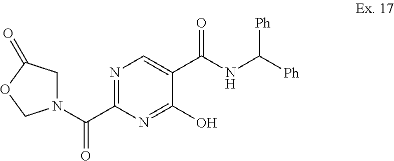 [SMILES: O=C1CN(C(=O)c2ncc(C(=O)NC(c3ccccc3)c3ccccc3)c(O)n2)CO1]

### Step A N-Benzhydryl-4-(benzyloxy)-2-(5-oxooxazolidine-3-carbonyl)pyrimidine-5-carboxamide

To a solution of 2-(5-(benzhydrylcarbamoyl)-4-(benzyloxy)pyrimidine-2-carboxamido)acetic acid (150 mg, 0.3 mmol) in DMF (3 mL) were added K₂CO₃ (125 mg, 0.9 mmol) and chloromethyl isopropyl carbonate (138 mg, 0.9 mmol). The mixture was stirred at room temperature overnight. The mixture was then poured into THF (20 mL), filtered and concentrated. The residue was purified by prep. HPLC (Instrument: Gilson® 215 Column: ASB C18 5 u 150*25 mm (Gilson, Inc., Middleton, Wis., USA), Mobile phase A: water (0.01 mol/L ammonium bicarbonate) Mobile phase B: acetonitrile) to afford N-benzhydryl-4-(benzyloxy)-2-(5-oxooxazolidine-3-carbonyl) pyrimidine-5-Carboxamide. ¹H NMR (CDCl₃, 400 MHz): δ 9.39 (s, 1H), 8.26 (d, J=8.0 Hz, 1H), 7.44-7.40 (m, 5H), 7.24-7.22 (m, 6H), 7.06-7.04 (m, 4H), 6.36 (d, J=8.0 Hz, 1H), 5.93 (s, 1H), 5.76 (s, 1H), 5.62 (s, 2H), 4.61 (s, 1H), 4.41 (s, 1H). LC/MS (m/z): 509 (M+H)⁺.

### Step B N-Benzhydryl-4-hydroxy-2-(5-oxooxazolidine-3-carbonyl)pyrimidine-5-carboxamide

To a solution of N-benzhydryl-4-(benzyloxy)-2-(5-oxooxazolidine-3-carbonyl) pyrimidine-5-carboxamide (70 mg, 0.14 mmol) in EtOAc (5 mL) was added Pd—C (10%, 50 mg). The mixture was stirred under hydrogen (balloon) at room temperature overnight. After filtration, the filtrate was concentrated to afford N-benzhydryl-4-hydroxy-2-(5-oxooxazolidine-3-carbonyl) pyrimidine-5-carboxamide, Ex. 17. ¹H NMR (CDCl₃, 400 MHz): δ 11.64 (brs, 1H), 9.92 (s, 1H), 9.02 (d, J=8.0 Hz, 1H), 7.34-7.23 (m, 10H), 6.46 (d, J=8.0 Hz, 1H), 6.10 (s, 2H), 5.63 (s, 1H), 4.84 (s, 1H), 4.31 (s, 1H). LC/MS (m/z): 419 (M+H). Human HIF-PHD2 IC₅₀: 15.1 nM.

### Example 18

### 1-((Isopropoxycarbonyl)oxy)ethyl 3-(5-(benzhydrylcarbamoyl)-4-hydroxypyrimidin-2-yl)-3-oxopropanoate

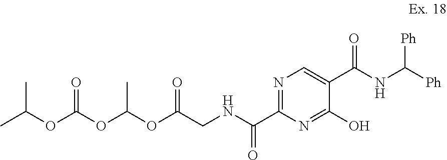 [SMILES: CC(C)OC(=O)OC(C)OC(=O)CNC(=O)c1ncc(C(=O)NC(c2ccccc2)c2ccccc2)c(O)n1]

### Step A 1-((Isopropoxycarbonyl)oxy)ethyl 2-(5-(benzhydrylcarbamoyl)-4-(benzyloxy)pyrimidine-2-carboxamido)acetate

To a solution of 2-(5-(benzhydrylcarbamoyl)-4-(benzyloxy)pyrimidine-2-carboxamido)acetic acid (0.2 g, 0.34 mmol) and 1-chloroethyl isopropyl carbonate (0.1 g, 0.67 mmol) in DMF (8 mL) was added K₂CO₃ (0.14 g, 1.0 mmol). The reaction mixture was stirred at 60° C. for 2 hours. When LCMS showed that the reaction was complete, the reaction mixture was filtered. The filtrate was concentrated under vacuum to afford the crude product. The crude product was purified by silica gel column chromatography (eluted with petroleum ether:EtOAc=2:3 vol.: vol.) to afford 1-((isopropoxycarbonyl)oxy)ethyl 2-(5-(benzhydrylcarbamoyl)-4-(benzyloxy)pyrimidine-2-carboxamido)acetate. ¹H NMR (CDCl₃, 400 MHz) δ 9.37 (s, 1H), 8.39 (s, 1H), 8.28 (d, J=8.2 Hz, 1H), 7.43-7.31 (m, 5H), 7.24-7.14 (m, 6H), 7.07-7.04 (m, 4H), 6.84 (q, J=5.2 Hz, 1H), 6.35 (d, J=8.2 Hz, 1H), 5.65 (s, 2H), 4.93-4.84 (m, 1H), 4.38 (dd, J=18.8, 6.0 Hz, 1H), 4.25 (dd, J=18.8, 4.8 Hz, 1H), 1.56 (d, J=5.3 Hz, 3H), 1.30 (d, J=5.6 Hz, 6H). LC/MS (m/z): 537 (M+H)⁺.

### Step B 1-((Isopropoxycarbonyl)oxy)ethyl 2-(5-(benzhydrylcarbamoyl)-4-hydroxypyrimidine-2-carboxamido)acetate

To a solution of 1-((isopropoxycarbonyl)oxy)ethyl 2-(5-(benzhydrylcarbamoyl)-4-(benzyloxy) pyrimidine-2-carboxamido)acetate (160 mg, 0.26 mmol) in EtOAc (20 mL) was added Pd/C (wet, 10%, 20 mg). The mixture was stirred under a hydrogenation atmosphere (balloon) for 2 h. When TLC showed the reaction was complete, the mixture was filtered. The filtrate was concentrated under vacuum to give 1-((isopropoxycarbonyl)oxy)ethyl 2-(5-(benzhydrylcarbamoyl)-4-hydroxypyrimidine-2-carboxamido)acetate, Ex. 18. ¹H NMR (CDCl₃, 400 MHz) δ 10.83 (brs, 1H), 9.85 (d, J=8.4 Hz, 1H), 8.98 (s, 1H), 8.18 (t, J=5.3 Hz, 1H), 7.34-7.29 (m, 8H), 7.26-7.21 (m, 2H), 6.83 (q, J=5.3 Hz, 1H), 6.43 (d, J=8.4 Hz, 1H), 4.94-4.84 (m, 1H), 4.33-4.15 (m, 2H), 1.55 (d, J=5.5 Hz, 3H), 1.30 (d, J=6.2 Hz, 6H). LC/MS (m/z): 559 (M+Na)⁺. Human HIF-PHD2 IC₅₀: 43.9 nM.

Example 19 in Table 4 was prepared following an analogous procedure to that described in Example 18 but by using the appropriate starting materials.

TABLE 4

| | | | MS m/z |
|---|---|---|---|
| | | | (M + 1)⁺ and |
| | | | human HIF- |
| Example | Name | Structure | PHD2 IC₅₀ |
| Ex. 19 | 1- ((ethoxycarbonyl) oxy)ethyl 2-(5- (benzhydryl- carbamoyl)-4- hydroxy- pyrimdine-2- carboxamido) acetate | 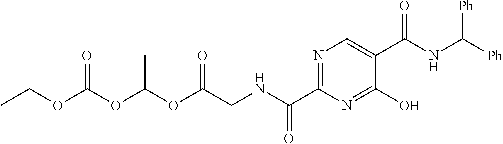 [SMILES: CCOC(=O)OC(C)OC(=O)CNC(=O)c1ncc(C(=O)NC(c2ccccc2)c2ccccc2)c(O)n1] | (M + 1)⁺ 522 IC₅₀ 32.2 nM |

### Example 20

### ((Isopropoxycarbonyl)oxy)methyl 2-(5-(benzhydrylcarbamoyl)-4-hydroxypyrimidine-2-carboxamido)acetate

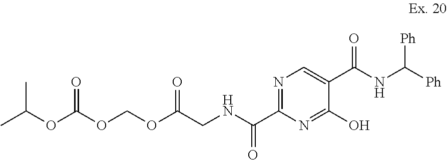 [SMILES: CC(C)OC(=O)OCOC(=O)CNC(=O)c1ncc(C(=O)NC(c2ccccc2)c2ccccc2)c(O)n1]

### Step A tert-Butyl 2-(5-(benzhydrylcarbamoyl)-4-(benzyloxy)pyrimidine-2-carboxamido)acetate

HATU (5.2 g, 13.7 mmol) in DMF (80 mL) was added dropwise via addition funnel over 30 min at RT to a solution of tert-butyl 2-aminoacetate hydrochloride (3.8 g, 22.8 mmol), 5-(benzhydrylcarbamoyl)-4-(benzyloxy)pyrimidine-2-carboxylic acid (5.0 g, 11.4 mmol) and TEA (10 mL, 45.6 mmol) in DMF (120 mL). The reaction mixture was stirred at RT for 5 hours, when LCMS showed the reaction completed. The reaction mixture was poured into 1000 mL H₂O. The precipitate was collected by filtration to afford the desired tert-butyl 2-(5-(benzhydrylcarbamoyl)-4-(benzyloxy)pyrimidine-2-carboxamido)acetate. ¹H NMR (CDCl₃, 400 MHz) δ 9.38 (s, 1H), 8.41-8.38 (m, 1H), 8.28 (d, J=8.0 Hz, 1H), 7.41-7.33 (m, 5H), 7.23-7.21 (m, 6H), 7.06-7.04 (m, 4H), 6.34 (d, J=8.0 Hz, 1H), 5.66 (s, 2H), 4.19 (d, J=5.3 Hz, 2H), 1.50 (s, 9H). LC/MS (m/z): 553 (M+H)+.

### Step B tert-Butyl 2-(5-(benzhydrylcarbamoyl)-4-hydroxypyrimidine-2-carboxamido)acetate

To a solution of tert-butyl 2-(5-(benzhydrylcarbamoyl)-4-(benzyloxy)pyrimidine-2-carboxamido) acetate (5.9 g, 10.7 mmol) in EtOAc (1500 mL) was added Pd/C (10%, 0.6 g). The reaction mixture was stirred under H₂ atmosphere for 2 hours at which time LCMS showed the starting material was consumed. The reaction mixture was filtered through a pad of CELITE, and the filtrate was concentrated under vacuum to afford the desired tert-butyl 2-(5-(benzhydrylcarbamoyl)-4-hydroxypyrimidine-2-carboxamido)acetate. ¹H NMR (Methanol-d₄, 400 MHz) δ 8.76 (s, 1H), 7.36-7.29 (m, 7H), 7.26-7.23 (m, 3H), 6.31 (d, J=7.7 Hz, 1H), 4.03 (s, 2H), 1.47 (s, 9H). LC/MS (m/z): 485 (M+Na)+.

### Step C 2-(5-(Benzhydrylcarbamoyl)-4-hydroxypyrimidine-2-carboxamido)acetic acid

A solution of tert-butyl 2-(5-(benzhydrylcarbamoyl)-4-hydroxypyrimidine-2-carboxamido) acetate (3.0 g, 6.5 mmol) in HCl (4M in dioxane, 50 mL) was stirred at RT for 6 hours. The reaction mixture was concentrated under vacuum to about 30 mL. About 80 mL petroleum ether was added to the residue. Resulting precipitate was collected by filtration to afford the desired product 2-(5-(benzhydrylcarbamoyl)-4-hydroxypyrimidine-2-carboxamido) acetic acid. ¹H NMR (DMSO-d₆, 400 MHz) δ 9.38 (t, J=6.0 Hz, 1H), 7.36-7.29 (m, 9H), 7.26-7.23 (m, 2H), 6.25 (d, J=7.9 Hz, 1H), 3.92 (d, J=6.0 Hz, 2H). LC/MS (m/z): 407 (M+H)⁺.

### Step D ((Isopropoxycarbonyl)oxy)methyl 2-(5-(benzhydrylcarbamoyl)-4-hydroxypyrimidine-2-carboxamido)acetate

To a solution of chloromethyl isopropyl carbonate (0.15 g, 0.98 mmol) in DMF (8 mL) was added 2-(5-(benzhydrylcarbamoyl)-4-hydroxypyrimidine-2-carboxamido) acetic acid (0.20 g, 0.49 mmol), followed by K₂CO₃ (0.203 g, 1.47 mmol). The reaction mixture was heated to 40° C. and stirred for 2 hours. When LCMS showed the reaction was complete, the mixture was filtered through a pad of CELITE, and the filtrate was concentrated under vacuum to afford an oil that was further purified by preparative TLC to give ((isopropoxycarbonyl)oxy) methyl 2-(5-(benzhydrylcarbamoyl)-4-hydroxypyrimidine-2-carboxamido)acetate, Ex. 20. ¹H NMR (CDCl₃, 400 MHz) 8.95 (s, 1H), 8.22 (s, 1H), 7.35-7.28 (m, 8H), 7.22-7.16 (m, 2H), 6.40 (d, J=7.1 Hz, 1H), 5.80 (s, 2H), 4.95-4.90 (m, 1H), 4.24 (s, 2H), 1.30 (d, J=4.6 Hz, 6H). LC/MS (m/z): 545 (M+Na)⁺. Human HIF-PHD2 IC₅₀: 41.0 nM.

Examples 21-22 in Table 5 were prepared following an analogous procedure to that described in Example 20 but by using the appropriate starting materials.

TABLE 5

| | | | MS m/z |
|---|---|---|---|
| | | | (M + 1)⁺ and |
| | | | human HIF- |
| Example | Name | Structure | PHD2 IC₅₀ |
| Ex. 21 | (2-(5- (benzhydrylcarbamoyl)- 4-hydroxypyrimidine-2- carboxamido)acetoxy) methyl isobutyrate | 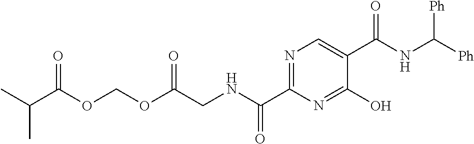 [SMILES: CC(C)C(=O)OCOC(=O)CNC(=O)c1ncc(C(=O)NC(c2ccccc2)c2ccccc2)c(O)n1] | (M + 1)⁺ 507 IC₅₀ 35.0 nM |
| Ex. 22 | (2-(5- (benzhydrylcarbamoyl)- 4-hydroxypyrimidine-2- carboxamido)acetoxy) methyl propionate | 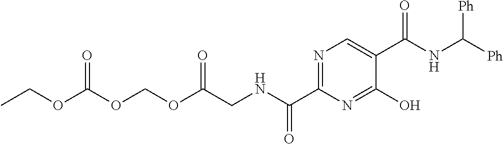 [SMILES: CCOC(=O)OCOC(=O)CNC(=O)c1ncc(C(=O)NC(c2ccccc2)c2ccccc2)c(O)n1] | (M + 1)⁺ 509 IC₅₀ 14.7 nM |

### Example 23

### (2R)-1-(Benzyloxy)-1-oxopropan-2-yl 3-(5-(benzhydrylcarbamoyl)-4-hydroxypyrimidine-2-carboxamido)-2-hydroxypropanoate

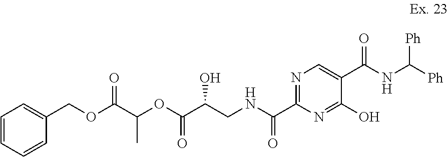 [SMILES: CC(OC(=O)[C@H](O)CNC(=O)c1ncc(C(=O)NC(c2ccccc2)c2ccccc2)c(O)n1)C(=O)OCc1ccccc1]

### Step A (R)-3-(5-(benzhydrylcarbamoyl)-4-hydroxypyrimidine-2-carboxamido)-2-hydroxypropanoic acid

To a solution of (R)-ethyl 3-(5-(benzhydrylcarbamoyl)-4-hydroxypyrimidine-2-carboxamido)-2-hydroxypropanoate (600 mg, 1.3 mmol) in THF (25 mL) was added aq. LiOH (1M, 6.5 mL, 6.5 mmol) at RT. The mixture was stirred at RT for 30 min. When TLC showed the reaction completed, the mixture was concentrated to remove THF, and the aqueous residue was acidified with 5% hydrochloric acid to pH 2. The precipitate was collected by filtration to give (R)-3-(5-(benzhydrylcarbamoyl)-4-hydroxypyrimidine-2-carboxamido)-2-hydroxypropanoic acid as a solid. ¹H NMR (DMSO-d₆, 400 MHz) δ 10.42 (br, 1H), 8.95 (s, 1H), 8.51 (br, 1H), 6.27 (d, J=7.6 Hz, 1H), 5.62 (br, 1H), 4.24-4.21 (m, 1H), 3.53-3.51 (m, 2H). LC/MS (m/z): 437 (M+H)⁺.

### Step B (2R)-1-(benzyloxy)-1-oxopropan-2-yl 3-(5-(benzhydrylcarbamoyl)-4-hydroxypyrimidine-2-carboxamido)-2-hydroxypropanoate

To a solution of (R)-3-(5-(benzhydrylcarbamoyl)-4-hydroxypyrimidine-2-carboxamido)-2-hydroxypropanoic acid (50 mg, 0.11 mmol) in CHCl₃ (3.0 mL) were added tris(2-(2-methoxyethoxy)ethyl)amine (140 mg, 0.44 mmol) and benzyl 2-bromopropanoate (50 mg, 0.22 mmol) at room temperature. The reaction mixture was stirred at 100° C. for 50 hours. The mixture was concentrated under vacuum to remove CHCl₃. The residue was purified by prep. HPLC (Instrument: Gilson 281; Column: Gemini® 150*23.5 mm*10 um; Mobile phase A: water (0.025% FA, V/V); Mobile phase B: acetonitrile; acetonitrile Conc: 32-62-16) to afford (2R)-1-(benzyloxy)-1-oxopropan-2-yl 3-(5-(benzhydrylcarbamoyl)-4-hydroxypyrimidine-2-carboxamido)-2-hydroxypropanoate as a solid, Ex. 23. ¹H NMR (DMSO-d₆, 400 MHz) δ 7.35-7.24 (m, 15H), 6.25 (d, J=8.0 Hz, 1H), 5.93-5.91 (m, 1H), 5.14-5.11 (m, 2H), 5.04-5.02 (m, 1H), 4.37-4.31 (m, 1H), 3.56-3.41 (m, 1H), 1.43-1.36 (m, 3H). LC/MS (m/z): 599 (M+H)+. Human HIF-PHD2 IC50: 11.2 nM.

Example 24 in Table 6 was prepared following analogous procedures to those described in Example 22 but by using the appropriate starting materials.

TABLE 6

| | | | MS m/z |
|---|---|---|---|
| | | | (M + 1)⁺ and |
| | | | human HIF- |
| Example | Name | Structure | PHD2 IC₅₀ |
| Ex. 24 | (2R)-2-ethoxy-2-oxo-1- phenylethyl 3-(5- (benzhydrylcarbamoyl)- 4-hydroxypyrimidine-2- carboxamido)-2- hydroxypropanoate | 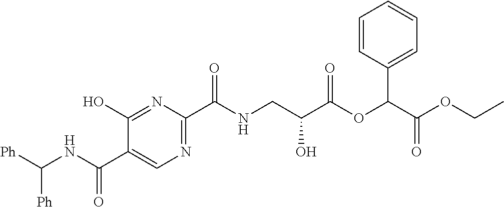 [SMILES: CCOC(=O)C(OC(=O)[C@H](O)CNC(=O)c1ncc(C(=O)NC(c2ccccc2)c2ccccc2)c(O)n1)c1ccccc1] | (M + 1)⁺ 599 IC₅₀ 326.1 nM |

### Example 25

### (2R)-2-Hydroxycyclohexyl 3-(5-(benzhydrylcarbamoyl)-4-hydroxypyrimidine-2-carboxamido)-2-hydroxypropanoate

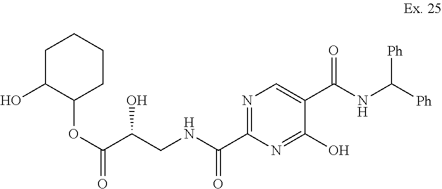 [SMILES: O=C(NC[C@@H](O)C(=O)OC1CCCCC1O)c1ncc(C(=O)NC(c2ccccc2)c2ccccc2)c(O)n1]

To a solution of sodium dodecane-1-sulfonate (940 mg, 3.4 mmol) in water (4 ml) were added K₂CO₃ (16 mg, 0.12 mmol), (R)-3-(5-(benzhydrylcarbamoyl)-4-hydroxypyrimidine-2-carboxamido)-2-hydroxypropanoic acid (50 mg, 0.12 mmol), 7-oxabicyclo[4.1.0] heptane (110 mg, 1.1 mmol) and erbium (III) triflate (70 mg, 0.12 mmol). The mixture was stirred at 50° C. for 48 h, then filtered upon cooling. The filtrate was purified by prep. HPLC (Instrument: Gilson® 281; Column: Gemini® 150*25 mm 10 u; Mobile phase A: water (0.025% FA, V/V); Mobile phase B: acetonitrile; acetonitrile Conc: 27-57-15) to afford (2R)-2-hydroxycyclohexyl 3-(5-(benzhydrylcarbamoyl)-4-hydroxypyrimidine-2-carboxamido)-2-hydroxypropanoate, Ex. 25, as a solid. ¹H NMR (DMSO-d₆, 400 MHz) δ 9.01 (d, J=8.0 Hz, 1H), 8.87 (s, 1H), 8.72 (t, J=5.2 Hz, 1H), 7.33-7.24 (m, 10H), 6.32 (d, J=8.0 Hz, 1H), 5.27-5.25 (m, 1H), 4.22-4.19 (m, 1H), 3.68-3.62 (m, 4H), 3.12 (br, 1H), 2.09-2.07 (m, 1H), 1.87-1.85 (m, 1H), 1.67-1.59 (m, 2H), 1.39-1.31 (m, 4H). LC/MS (m/z): 519 (M+H)⁺. Human HIF-PHD2 IC₅₀: 181.8 nM.

### Examples 26 and 27

### Ethyl 3-(5-(benzhydrylcarbamoyl)-4-hydroxypyrimidine-2-carboxamido)-2-hydroxy-2-methylpropanoate

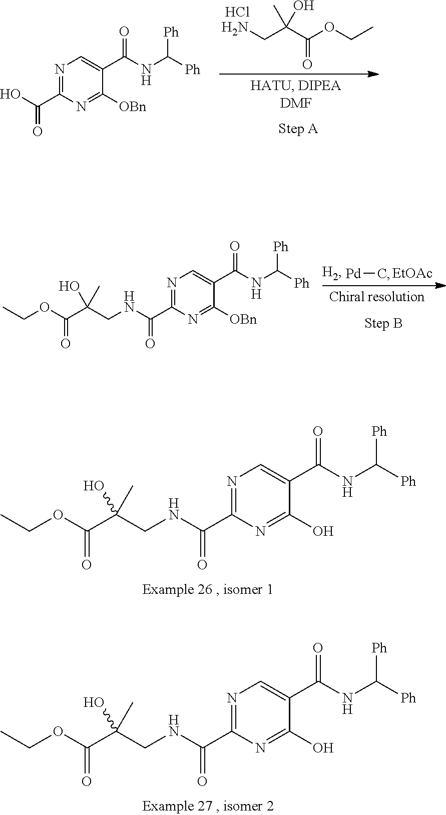

### Step A Ethyl 3-(5-(benzhydrylcarbamoyl)-4-(benzyloxy)pyrimidine-2-carboxamido)-2-hydroxy-2-methylpropanoate

To a 100 mL single-neck flask equipped with a stirring bar were added ethyl 3-amino-2-hydroxy-2-methylpropanoate hydrochloride (251 mg, 1.3 mmol) and DMF (8 mL). To this solution were added DIPEA (351 mg, 2.7 mmol), 5-(benzhydrylcarbamoyl)-4-(benzyloxy) pyrimidine-2-carboxylic acid (300 mg, 0.7 mmol) and HATU (517 mg, 1.3 mmol) at room temperature. The mixture was stirred at RT for 16 h, then poured into water and extracted with EtOAc (200 mL). The organic layer was washed with water and brine, dried over anhydrous sodium sulfate and concentrated under vacuum. The residue was purified by column chromatography on silica gel (eluted with Petroleum Ether/EtOAc 1:1-1:2) to afford ethyl 3-(5-(benzhydrylcarbamoyl)-4-(benzyloxy) pyrimidine-2-carboxamido)-2-hydroxy-2-methylpropanoate as a solid. ¹H NMR (CDCl₃, 400 MHz) δ 9.37 (s, 1H), 8.32-8.21 (m, 2H), 7.40-7.05 (m, 15H), 6.35 (d, J=8.0 Hz, 1H), 5.64 (s, 2H), 4.25 (q, J=7.2 Hz, 2H), 4.03-3.98 (m, 1H), 3.62 (br s, 1H), 3.61-3.58 (m, 1H), 1.48 (s, 3H), 1.29 (t, J=7.2 Hz, 3H). LC/MS (m/z): 569 (M+H)⁺.

### Step B Ethyl 3-(5-(benzhydrylcarbamoyl)-4-hydroxypyrimidine-2-carboxamido)-2-hydroxy-2-methylpropanoate

To a solution of ethyl 3-(5-(benzhydrylcarbamoyl)-4-(benzyloxy) pyrimidine-2-carboxamido)-2-hydroxy-2-methylpropanoate (200 mg, 0.35 mmol) in EtOAc (20 mL) was added Pd/C (Wet, 10%, 50 mg). The mixture was stirred under hydrogen atmosphere for 1 h. When TLC showed that the reaction was complete, the reaction mixture was filtered through a pad of CELITE, and the filtrate was concentrated under reduced pressure to afford ethyl 3-(5-(benzhydrylcarbamoyl)-4-hydroxypyrimidine-2-carboxamido)-2-hydroxy-2-ethylpropanoate as a solid. LC/MS (m/z): 479 (M+H)⁺.

Ethyl 3-(5-(benzhydrylcarbamoyl)-4-hydroxypyrimidine-2-carboxamido)-2-hydroxy-2-methyl propanoate (400 mg, 0.84 mmol) was dissolved in MeOH and resolved by SFC (Instrument: MG-II; Column: Chiralcel™ OD 250×30 mm I.D., 5 μm; Mobile phase: Supercritical CO₂/MeOH (0.1%) NH₃.H₂O=60/40 at 50 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm) to afford:

### Example 26

Peak 1 (RT=7.03 min) of ethyl 3-(5-(benzhydrylcarbamoyl)-4-(benzyloxy)pyrimidine-2-carboxamido)-2-hydroxypropanoate as a solid. ¹H NMR (CDCl₃, 400 MHz) δ 9.89 (d, J=8.0 Hz, 1H), 8.95 (s, 1H), 8.11 (t, J=7.6 Hz, 1H), 7.32-7.24 (m, 10H), 6.43 (d, J=8.0 Hz, 1H), 4.24 (q, J=7.2 Hz, 2H), 3.94-3.91 (m, 1H), 3.55 (br s, 1H), 3.54-3.50 (m, 1H), 1.45 (s, 3H), 1.28 (t, J=7.2 Hz, 3H). LC/MS (m/z): 479 (M+H)⁺. Human HIF-PHD2 IC₅₀: 940 nM.

### Example 27

Peak 2 (RT=7.43 min) as solid. ¹H NMR (CDCl₃, 400 MHz) δ 9.92 (d, J=8.4 Hz, 1H), 8.96 (s, 1H), 8.12 (t, J=7.6 Hz, 1H), 7.33-7.26 (m, 10H), 6.44 (d, J=8.4 Hz, 1H), 4.25 (q, J=7.2 Hz, 2H), 3.97-3.93 (m, 1H), 3.56 (br s, 1H), 3.55-3.51 (m, 1H), 1.46 (s, 3H), 1.29 (t, J=7.2 Hz, 3H). LC/MS (m/z): 479 (M+H)⁺. Human HIF-PHD2 IC₅₀: 879 nM.

### Example 28

### (Benzyl 3-(5-(benzhydrylcarbamoyl)-4-hydroxypyrimidine-2-carboxamido)propanoate

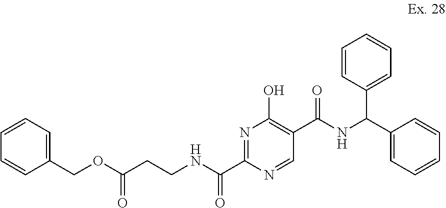 [SMILES: O=C(CCNC(=O)c1ncc(C(=O)NC(c2ccccc2)c2ccccc2)c(O)n1)OCc1ccccc1]

The title compound was prepared following a procedure analogous to that described in Example 1 using the appropriate starting materials. ¹H NMR (CDCl₃, 500 MHz) δ: 9.82 (m, 1H), 8.23 (m, 1H), 7.38 (m, 10H), 7.20 (m, 5H), 6.41 (d, 1H), 5.19 (s, 2H), 3.78 (d, 2H), 2.72 (d, 2H). LC/MS (m/z): 511 (M+H)⁺. Human HIF-PHD2 IC₅₀: 24.34 nM.

### Examples 29

### (1R,3R)-2-isopropoxy-2-oxoethyl 3-(5-(benzhydrylcarbamoyl)-4-hydroxypyrimidine-2-carboxamido)cyclobutanecarboxylate

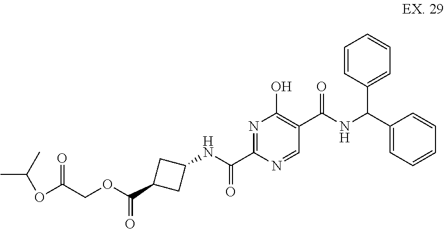 [SMILES: CC(C)OC(=O)COC(=O)[C@H]1C[C@H](NC(=O)c2ncc(C(=O)NC(c3ccccc3)c3ccccc3)c(O)n2)C1]

### Step A (1R,3R)-methyl 3-(5-(benzhydrylcarbamoyl)-4-(benzyloxy)pyrimidine-2-carboxamido)cyclobutanecarboxylate

To a solution of 5-(benzhydrylcarbamoyl)-4-(benzyloxy)pyrimidine-2-carboxylic acid (550 mg, 1.252 mmol) and (1R,3R)-methyl 3-aminocyclobutanecarboxylate hydrochloride (249 mg, 1.502 mmol) in DMF (6 mL) was added DIEA (0.656 ml, 3.75 mmol), followed by HATU (523 mg, 1.377 mmol). The reaction was stirred at room temperature for 3 hr. The mixture was diluted with water (30 mL) and extracted with EtOAc (100 mL). The organic layer was washed with water (30 mL×2) and brine (30 mL), dried with Na₂SO₄ and concentrated to afford crude product. Purification by silica column (ISCO, 24 g redflash column, 30 mL/min, 0%-100% EtOAc in Hexane) afforded desired product as a solid. LC/MS (m/z): 551 (M+H)⁺.

### Step B (1R,3R)-methyl 3-(5-(benzhydrylcarbamoyl)-4-hydroxypyrimidine-2-carboxamido)cyclobutanecarboxylate

To step A product (120 mg, 0.218 mmol) was added 4 M HCl in dioxane (2.070 ml, 8.28 mmol). The mixture was stirred at room temperature for 2 hr. The solvent was removed under vacuum to afford crude product which was used in the next step without further purification. LC/MS (m/z): 483 (M+23)⁺.

### Step C (1R,3R)-3-(5-(benzhydrylcarbamoyl)-4-hydroxypyrimidine-2-carboxamido)cyclobutanecarboxylic acid

The Step B product (350 mg, 0.760 mmol) in THF (2 mL) was added 2 M LiOH (3.80 ml, 7.60 mmol). The mixture was stirred at room temperature for 2 hr. The solvent was removed and the mixture was acidified with 2 M HCl to adjust pH to ˜2. The solid was precipitated and filtered. The collected solid was washed with Ether (×3) and Hexane (×3). The solid was suspended in CH₃CN (3 mL) and water (3 mL) that was lyophilized to obtain the desired product as a solid. LC/MS (m/z): 469 (M+23)+.

### Step D (1R,3R)-2-isopropoxy-2-oxoethyl 3-(5-(benzhydrylcarbamoyl)-4-hydroxypyrimidine-2-carboxamido)cyclobutanecarboxylate

To the Step C product (30 mg, 0.067 mmol) and DMF (0.6 mL) was added Cs₂CO₃ (21.70 mg, 0.067 mmol) followed by isopropyl 2-bromoacetate (8.62 μl, 0.067 mmol). The reaction was stirred at room temperature for 1 hr. To the mixture were added water and TFA to quench the reaction. The mixture was dissolved in 0.5 mL CH₃CN. Purification with HPLC (reverse phase, C₁₈ column), eluted with 30% CH₃CN (0.1% TFA) in H₂O (with 0.1% TFA) to 90% 30% CH₃CN (0.1% TFA) in H₂O (with 0.1% TFA) afforded the title compound, Ex. 29, as a solid after lyophilization. ¹H NMR (CD₃OD, 500 MHz) δ: 7.37 (m, 6H), 7.22 (m, 3H), 6.82 (d, 2H), 6.21 (d, 1H), 5.01 (m, 1H), 4.61 (s, 2H), 3.78 (s, 2H), 3.70 (m, 2H). 2.78 (m, 2H), 1.23 (d, 1H), 1.20 (d, 6H). LC/MS (m/z): 569.12 (M+23)⁺. Human HIF-PHD2 IC₅₀ 171 nM.

### Example 30

### 2-Isopropoxy-2-oxoethyl 3-(5-(benzhydrylcarbamoyl)-4-hydroxypyrimidine-2-carboxamido)-2-methylpropanoate

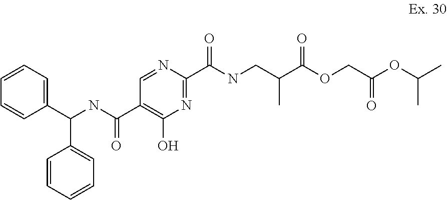 [SMILES: CC(C)OC(=O)COC(=O)C(C)CNC(=O)c1ncc(C(=O)NC(c2ccccc2)c2ccccc2)c(O)n1]

### Step A Methyl 3-(5-(benzhydrylcarbamoyl)-4-(benzyloxy)pyrimidine-2-carboxamido)-2-methylpropanoate

To a solution of 5-(benzhydrylcarbamoyl)-4-(benzyloxy)pyrimidine-2-carboxylic acid (600 mg, 1.4 mmol) in DMF (10 mL) was added methyl 3-amino-2-methylpropanoate hydrochloride (420 mg, 2.7 mmol), TEA (270 mg, 2.6 mmol) and HATU (610 mg, 1.6 mmol). The mixture was stirred at room temperature overnight. When TLC showed the reaction completed, the reaction mixture was washed with water (20 mL) and then extracted with EtOAc (40 mL). The organic layer was washed with brine (20 mL), dried over sodium sulfate, filtered and concentrated under vacuum. The residue was purified by prep. TLC (DCM/MeOH=15:1) to afford methyl 3-(5-(benzhydrylcarbamoyl)-4-(benzyloxy)pyrimidine-2-carbox amido)-2-methylpropanoate as a solid. ¹H NMR (CDCl₃, 400 MHz) δ 9.30 (s, 1H), 8.38 (t, J=6.2 Hz, 1H), 8.23 (d, J=8.0 Hz, 1H), 7.36-7.27 (m, 5H), 7.18-7.13 (m, 6H), 7.02-6.96 (m, 4H), 6.29 (d, J=8.0 Hz, 1H), 5.61 (s, 2H), 4.05 (q, J=7.1 Hz, 1H), 3.65 (s, 3H), 3.53 (ddd, J=6.2, 7.8, 13.8 Hz, 1H), 2.79 (dt, J=4.6, 7.5 Hz, 1H), 1.22-1.19 (m, 3H). LC/MS (m/z): 539 (M+H)⁺.

### Step B 3-(5-(Benzhydrylcarbamoyl)-4-hydroxypyrimidine-2-carboxamido)-2-methylpropanoic acid

To a solution of Step A product (410 mg, 0.8 mmol) in EtOAc (20 mL) was added Pd/C (Wet, 10%, 50 mg). The mixture was stirred under hydrogen atmosphere for 1 h. When TLC showed that the reaction completed, the reaction mixture was filtered through a pad of CELITE, and the filtrate was concentrated under reduced pressure to afford methyl 3-(5-(benzhydrylcarbamoyl)-4-(benzyloxy)pyrimidine-2-carboxamido)-2-methyl propanoate as a solid. LC/MS (m/z): 439 (M+H)⁺. To the above product (100 mg, 0.22 mmol) in THF (3 mL) was added aq. LiOH (0.5 mL, 0.5 mmol). After addition, the reaction mixture was allowed to stir at rt for 30 min, when TLC showed the reaction was completed. The mixture was acidified with aq. HCl (5%) to pH 3-4. The precipitate was collected by filtration to afford 2-carboxamido)-2-methylpropanoic acid as a solid. LC/MS (m/z): 435 (M+H)⁺.

### Step C 3-(5-(benzhydrylcarbamoyl)-4-hydroxypyrimidine-2-carboxamido)-2-methyl propanoic acid

The product of Step B was resolved by SFC (SFC condition: Chiralpak™ AD-H® 250*4.6 mm I.D., 5 um; 40% iPrOH (0.05% DEA) in CO2; 2.35 mL/min 220 nm) to give:

Isomer 1 (RT 2.61 min): ¹H NMR (Methanol-d₄, 400 MHz) δ 8.65 (br d, J=8.0 Hz, 1H), 7.24-7.16 (m, 10H), 6.22 (d, J=8.0 Hz, 1H), 3.52-3.47 (m, 1H), 3.42-3.37 (m, 1H), 2.71-2.66 (m, 1H), 1.10 (d, J=7.2 Hz, 3H). LC/MS (m/z): 435 (M+H)⁺. and

Isomer 2 (RT 2.99 min): ¹H NMR (Methanol-d₄, 400 MHz) δ 8.73 (br d, J=8.0 Hz, 1H), 7.34-7.24 (m, 10H), 6.30 (d, J=8.0 Hz, 1H), 3.61-3.56 (m, 1H), 3.51-3.36 (m, 1H), 2.79-2.74 (m, 1H), 1.20 (d, J=7.2 Hz, 3H). LC/MS (m/z): 435 (M+H)⁺.

### Step D 2-Isopropoxy-2-oxoethyl 3-(5-(benzhydrylcarbamoyl)-4-hydroxypyrimidine-2-carboxamido)-2-methylpropanoate

To isomer 2 of Step C (35 mg, 0.081 mmol) in DMF (0.6 mL) was added Cs₂CO₃ (26.2 mg, 0.081 mmol), then followed by isopropyl 2-bromoacetate (10.42 μl, 0.081 mmol). The reaction mixture was stirred at room temperature for 1 hr. To the mixture were added water and TFA to quench the reaction. The mixture was dissolved in 0.5 mL CH₃CN. Purification with HPLC (reverse phase, C₁₈ column), eluted with 30% CH₃CN (0.1% TFA) in H₂O (with 0.1% TFA) to 90% 30% CH₃CN (0.1% TFA) in H₂O (with 0.1% TFA) afforded the title compound, Ex. 30, as a solid after lyophilization. ¹H NMR (CD₃OD, 500 MHz) δ: 8.78 (s, 1H), 7.32 (m, 10H), 6.32 (s, 1H), 5.01 (m, 1H), 4.61 (s, 2H), 3.62 (m, 1H), 3.59 (m, 1H), 2.98 (m, 1H), 1.23 (m, 9H). LC/MS (m/z): 557 (M+23)⁺. Human HIF-PHD2 IC₅₀ 179.3 nM.

The carboxylic acid pro-drugs described in this application can increase absorption and oral bioavailability of parent acids in rats. As a comparison, the PK profile pro-drug of Example 3 and its parent acid is shown in Table 7.

TABLE 7
Rat^{a} pharmacokinetic data^{b} of pro-
drug and its parent carboxylic acid

| | Clₚ | T_{1/2} | PO AUC_{N} | Fₒᵣₐₗ |
|---|---|---|---|---|
| Compound | (mL/min/kg) | (hr) | (μM · h · kg/mg) | (%) |
| Example 3 | 5.05 | 1.28 | 3.56 | 51.4 |
| Parent acid^{c} | 25.4 | 4.28 | 0.29 | 16.3 |

^{a}Wistar Han (SD) rat was used.
^{b}Plasma clearance (Clp) and half life (T½) calculated following 1 mg/kg IV dose. Normalized oral exposure (PO AUCN) and oral bioavailability (Foral) calculated following 2 mg/kg PO dose.
^{c}The parent acid was obtained by hydrolysis of Example 3 pro-drug with aqueous LiOH/THF. MS m/z (M + 1)⁺ 447.

### Biological Assays

The exemplified compounds of the present invention have been found to inhibit the hydroxylation of a HIF peptide by PHD2 and exhibit IC₅₀ values ranging between 0.1 nanomolar to 10 micromolar. Select examples of assays that may be used to detect favorable activity are disclosed in the following publications: Oehme, F., et al., Anal. Biochem. 330:74-80 (2004); Hirsilä, M, et al., J. Bio. Chem. 278 (33): 30772-30780 (2005); Hyunju, C., et al., Biochem. Biophys. Res. Comm. 330 275-280 (2005); and Hewitson, K. S., et al., Methods in Enzymology, (Oxygen Biology and Hypoxia); Elsevier Publisher (2007), pg. 25-42 (ISSN: 0076-6879).

The biological activity of the present compounds may be evaluated using assays described herein below:

To each well of a 384-well plate, 1 μL of test compounds in DMSO (final concentration ranging from 0.3 nM to 10 uM) were added into 20 μl of assay buffer (50 mM Tris pH 7.4/0.01% Tween-20/0.1 mg/ml bovine serum albumin/10 M ferrous sulfate/1 mM sodium ascorbate/20 μg/ml catalase) containing 0.15 μg/ml FLAG-tagged full length PHD2 expressed in and purified from baculovirus-infected Sf9 cells. After a 5 min preincubation at room temperature, the enzymatic reactions were initiated by the addition of 4 μL of substrates {final concentrations of 0.2 μM 2-oxoglutarate and 0.5 μM HIF-1α peptide biotinyl-DLDLEMLAPYIPMDDDFQL (SEQ ID NO: 1)}. After incubation for 45 minutes at room temperature, the reactions were terminated by the addition of a 25 μL quench/detection mix to a final concentration of 1 mM ortho-phenanthroline, 0.1 mM EDTA, 0.5 nM anti-(His)6 LANCE reagent (Perkin-Elmer Life Sciences), 100 nM AF647-labeled streptavidin (Invitrogen), and 2 μg/ml (His)6-VHL complex {S. Tan Protein Expr. Purif. 21, 224-234 (2001)} and the signals were developed for 30 minutes at room temperature. The ratio of time resolved fluorescence signals at 665 and 620 nm was determined, and percent inhibition was calculated relative to the high control samples (DMSO treated) run in parallel, after background subtraction.

Inhibition of the catalytic activity of HIF-PHD1 and HIF-PHD3 can be determined similarly, except for HIF-PHD3, final concentrations of 4 M 2-oxoglutarate is used during the reaction.

1-10. (canceled)

11. A compound which is:
Methyl 3-(5-(benzhydrylcarbamoyl)-4-hydroxypyrimidine-2-carboxamido)propanoate;
2-morpholinoethyl 3-(5-(benzhydrylcarbamoyl)-4-hydroxypyrimidine-2-carboxamido)propanoate;
ethyl 1-((5-(benzhydrylcarbamoyl)-4-hydroxypyrimidine-2-carboxamido)methyl)cyclopropanecarboxylate;
ethyl 3-(5-(benzhydrylcarbamoyl)-4-hydroxypyrimidine-2-carboxamido)-2-hydroxy-2-methylpropanoate;
(R)-ethyl 3-(5-(benzhydrylcarbamoyl)-4-hydroxypyrimidine-2-carboxamido)-2-hydroxypropanoate;
Isopropyl 3-(5-(benzhydrylcarbamoyl)-4-hydroxypyrimidine-2-carboxamido)propanoate;
Acetoxymethyl 3-(5-(benzhydrylcarbamoyl)-4-hydroxypyrimidine-2-carboxamido)propanoate;
((isopropoxycarbonyl)oxy)methyl 3-(5-(benzhydrylcarbamoyl)-4-hydroxypyrimidine-2-carboxamido)propanoate;
1-((ethoxycarbonyl)oxy) ethyl 3-(5-(benzhydrylcarbamoyl)-4-hydroxypyrimidine-2-carboxamido)propanoate;
1-((isopropoxycarbonyl) oxy)ethyl 3-(5-(benzhydrylcarbamoyl)-4-hydroxypyrimidine-2-carboxamido)propanoate;
1-(((cyclohexyloxy) carbonyl)oxy)ethyl 3-(5-(benzhydrylcarbamoyl)-4-hydroxypyrimidine-2-carboxamido)propanoate;
((ethoxycarbonyl)oxy) methyl 3-(5-(benzhydrylcarbamoyl)-4-hydroxypyrimidine-2-carboxamido)propanoate;
2-(Dimethylamino)-2-oxoethyl 3-(5-(benzhydrylcarbamoyl)-4-hydroxypyrimidine-2-carboxamido)propanoate;
N-Benzhydryl-4-hydroxy-2-(6-oxo-1,3-oxazinane-3-carbonyl)pyrimidine-5-carboxamide;
2,3-Dihydro-1H-inden-5-yl 2-(5-(benzhydrylcarbamoyl)-4-hydroxypyrimidine-2-carboxamido)acetate;
2-(dimethylamino)-2-oxoethyl 2-(5-(benzhydrylcarbamoyl)-4-hydroxypyrimidine-2-carboxamido)acetate;
N-Benzhydryl-4-hydroxy-2-(5-oxooxazolidine-3-carbonyl)pyrimidine-5-carboxamide;
1-((Isopropoxycarbonyl)oxy)ethyl 3-(5-(benzhydrylcarbamoyl)-4-hydroxypyrimidin-2-yl)-3-oxopropanoate;
1-((ethoxycarbonyl)oxy)ethyl 2-(5-(benzhydrylcarbamoyl)-4-hydroxypyrimidine-2-carboxamido)acetate;
((Isopropoxycarbonyl)oxy)methyl 2-(5-(benzhydrylcarbamoyl)-4-hydroxypyrimidine-2-carboxamido)acetate;
(2-(5-(benzhydrylcarbamoyl)-4-hydroxypyrimidine-2-carboxamido)acetoxy)methyl isobutyrate;
(2-(5-(benzhydrylcarbamoyl)-4-hydroxypyrimidine-2-carboxamido)acetoxy)methyl propionate;
(2R)-1-(Benzyloxy)-1-oxopropan-2-yl 3-(5-(benzhydrylcarbamoyl)-4-hydroxypyrimidine-2-carboxamido)-2-hydroxypropanoate;
(2R)-2-ethoxy-2-oxo-1-phenylethyl 3-(5-(benzhydrylcarbamoyl)-4-hydroxypyrimidine-2-carboxamido)-2-hydroxypropanoate;
(2R)-2-Hydroxycyclohexyl 3-(5-(benzhydrylcarbamoyl)-4-hydroxypyrimidine-2-carboxamido)-2-hydroxypropanoate;
Ethyl 3-(5-(benzhydrylcarbamoyl)-4-hydroxypyrimidine-2-carboxamido)-2-hydroxy-2-methylpropanoate;
(Benzyl 3-(5-(benzhydrylcarbamoyl)-4-hydroxypyrimidine-2-carboxamido)propanoate;
(1R,3R)-2-isopropoxy-2-oxoethyl 3-(5-(benzhydrylcarbamoyl)-4-hydroxypyrimidine-2-carboxamido)cyclobutanecarboxylate; or
2-Isopropoxy-2-oxoethyl 3-(5-(benzhydrylcarbamoyl)-4-hydroxypyrimidine-2-carboxamido)-2-methylpropanoate; or
a stereoisomer thereof or a pharmaceutically acceptable salt thereof.

12. A compound of claim 11 or a pharmaceutically acceptable salt thereof, for use as a medicament.

13. A compound of claim 11 or a pharmaceutically acceptable salt thereof, for the treatment of conditions mediated by HIF prolyl hydroxylase.

14. A pharmaceutical composition comprising a compound of claim 11 and pharmaceutically acceptable carrier.

15. A method of enhancing endogenous production of erythropoietin in a mammal which comprises administering to the mammal an amount of a compound of claim 11, or a pharmaceutically acceptable salt or solvate thereof, that is effective for enhancing endogenous production of erythropoietin.

16. A method for the treatment of anemia in a mammal which comprises administering to the mammal an effective amount of a compound of claim 11, or a pharmaceutically acceptable salt or solvate thereof.

17. A method for the prevention of anemia in a mammal which comprises administering to the mammal an effective amount of a compound of claim 11, or a pharmaceutically acceptable salt or solvate thereof.

18. Use of a compound of claim 11, or a pharmaceutically acceptable salt or solvate thereof, in the manufacture of medicaments for the treatment of conditions mediated by HIF prolyl hydroxylase.
